(19) United States
(12) Patent Application Publication
(10) Pub. No.: US 2012/0156737 A1
(43) Pub. Date: Jun. 21, 2012

(54) PREPARATION OF ALPHA-KETOPIMELIC ACID

(75) Inventors: Petronella Catharina Raemakers-Franken, KJ Budel (NL); Martin Schurmann, Julich (DE); Axel Christoph Trefzer, Leidschendam (DE); Stefaan Marie Andre De Wildeman, Maasmechelen (BE)

(73) Assignee: DSM IP ASSETS B.V., TE Heerlen (NL)

(21) Appl. No.: 13/255,161

(22) PCT Filed: Mar. 11, 2010
(86) PCT No.: PCT/NL2010/050126

§ 371 Date: Jan. 6, 2012

(30) Foreign Application Priority Data
Mar. 11, 2009 (EP) 09154840.4
Sep. 11, 2009 (EP) 09170092.2
Dec. 22, 2009 (EP) 09180441.9

(51) Int. Cl. C12P 7/50; C12P 13/00; C12P 17/10; C12N 1/19; C12N 1/21; C12P 7/40; C12N 1/15

(52) U.S. Cl. 435/121; 435/143; 435/136; 435/128; 435/254.5; 435/254.3; 435/254.11; 435/254.21; 435/254.2; 435/254.23; 435/252.33; 435/252.3; 435/252.34

(57) ABSTRACT

The invention relates to a method for preparing alpha-ketopimelic acid, comprising converting alpha-ketoglutaric acid into alpha-ketoadipic acid and converting alpha-ketoadipic acid into alpha-ketopimelic acid, wherein at least one of these conversions is carried out using a heterologous biocatalyst. The invention further relates to a heterologous cell, comprising one or more heterologous nucleic acid sequences encoding one or more heterologous enzymes capable of catalysing at least one reaction step in the preparation of alpha-ketopimelic acid from alpha-ketoglutaric acid.

The invention relates to a method for preparing alpha-ketopimelic acid (hereinafter also referred to as ‘AKP’; AKP is also known as 2-oxo-heptanedioic acid). The invention further relates to a method for preparing 5-formylpentanoic acid (hereinafter also referred to as ‘5-EVA’) and to a method for preparing 6-aminocaproic acid (hereinafter also referred to as ‘6-ACA’). The invention also relates to a method for preparing diaminohexane (also known as 1,6-hexanediamine). The invention further relates to a heterologous cell which may be used in a method according to the invention. The invention further relates to the use of a heterologous cell in the preparation of ε-caprolactam (hereafter referred to as ‘caprolactam’), 6-aminocaproic acid or diaminohexane.

Diaminohexane is inter alia used for the production of polyamides such as nylon 6,6. Other uses include uses as starting material for other building blocks (e.g. hexamethylene diisocyanate) and as crosslinking agent for epoxides. A Known preparation method proceeds from acrylonitrile via adiponitrile.

Caprolactam is a lactam which may be used for the production of polyamide, for instance nylon-6 or nylon-6,12 (a copolymer of caprolactam and laurolactam). Various manners of preparing caprolactam from bulk chemicals are known in the art and include the preparation of caprolactam from cyclohexanone, toluene, phenol, cyclohexanol, benzene or cyclohexane. These intermediate compounds are generally obtained from mineral oil. In view of a growing desire to prepare materials using more sustainable technology it would be desirable to provide a method wherein caprolactam is prepared from an intermediate compound that can be obtained from a biologically renewable source or at least from an intermediate compound that is converted into caprolactam using a biochemical method. Further, it would be desirable to provide a method that requires less energy than conventional chemical processes making use of bulk chemicals from petrochemical origin.

It is known to prepare caprolactam from 6-ACA, e.g. as described in U.S. Pat. No. 6,194,572. As disclosed in WO 2005/068643, 6-ACA may be prepared biochemically by converting 6-aminohex-2-enoic acid (6-AHEA) in the presence of an enzyme having α,β-enoate reductase activity. The 6-AHEA may be prepared from lysine, e.g. biochemically or by pure chemical synthesis. Although the preparation of 6-ACA via the reduction of 6-AHEA is feasible by the methods disclosed in WO 2005/068643, the inventors have found that—under the reduction reaction conditions—6-AHEA may spontaneously and substantially irreversibly cyclise to form an undesired side-product, notably β-homoproline. This cyclisation may be a bottleneck in the production of 6-ACA, and may lead to a considerable loss in yield.

The inventors have realised that it is possible to prepare 6-ACA from AKP. AKP can be prepared chemically, e.g. based on a method as described by H. Jäger et al. Chem. Ber. 1959, 92, 2492-2499. AKP can be prepared by alkylating cyclopentanone with diethyl oxalate using sodium ethoxide as a base, refluxing the resultant product in a strong acid (2 M HCl) and recovering the product, e.g. by crystallisation from toluene. However, as indicated above, there is a growing desire to prepare materials using more sustainable technology. Thus, the inventors realised it would be desirable to provide a method wherein AKP is prepared from an intermediate compound that can be obtained from a biologically renewable source.

It is an object of the invention to provide a novel method for preparing AKP, which may be used, in particular, for the preparation of 6-ACA, diaminohexane or another compound.

It is further an object to provide a novel biocatalyst, suitable for catalysing one or more reaction step in a method for preparing AKP.

One or more further objects which may be solved in accordance with the invention will follow from the description below.

The inventors have realised it is possible to prepare AKP using a specific biocatalyst.

Accordingly, the present invention relates to a method for preparing AKP, comprising converting alpha-ketoglutaric acid (AKG) into alpha-ketoadipic acid (AKA) and converting alpha-ketoadipic acid into alpha-ketopimelic acid, wherein at least one of these conversions is carried out using a biocatalyst, in particular a heterologous biocatalyst.

The AKP may for instance be used as an intermediate in the preparation of 5-formylpentanoic acid (5-FVA).

Accordingly, the invention further relates to a method for preparing 5-FVA comprising biocatalytically decarboxylating AKP prepared in a method according to the invention thereby forming 5-FVA.

The 5-FVA is for instance a suitable intermediate compound for preparing 6-ACA, caprolactam or diaminohexane.

The AKP may for instance be used as an intermediate in the preparation of alpha amino-pimelic acid (AAP).

Accordingly, the invention further relates to a method for preparing AAP comprising biocatalytically transaminating AKP prepared in a method according to the invention, thereby forming AAP.

The AAP is for instance a suitable intermediate compound for preparing 6-ACA, or caprolactam.

6-ACA may for instance be converted into caprolactam or into diaminohexane.

The invention further provides a heterologous cell, comprising one or more heterologous nucleic acid sequences encoding one or more heterologous enzymes capable of catalysing at least one reaction step in the preparation of alpha-ketopimelic acid from alpha-ketoglutaric acid.

Such cell may in particular be used as a biocatalyst in a method for preparing at least one compound selected from the group of AKP, 5-FVA, 6-ACA, diaminohexane and caprolactam.

In accordance with the invention, no problems have been noticed with respect to an undesired cyclisation of an intermediate product, when forming 6-ACA and optionally caprolactam, resulting in a loss of yield.

It is envisaged that a method of the invention allows a comparable or even better yield than the method described in WO 2005/68643. It is envisaged that a method of the invention may in particular be favourable if use is made of a living organism—in particular in a method wherein growth and maintenance of the organism is taken into account.

It is further envisaged that in an embodiment of the invention the productivity of 6-ACA (g/l.h formed) in a method of the invention may be improved.

The term “or” as used herein is defined as “and/or” unless specified otherwise.

The term “a” or “an” as used herein is defined as “at least one” unless specified otherwise.

When referring to a noun (e.g. a compound, an additive, etc.) in the singular, the plural is meant to be included. Thus, when referring to a specific moiety, e.g. “compound”, this means “at least one” of that moiety, e.g. “at least one compound”, unless specified otherwise.

When referred herein to carboxylic acids or carboxylates, e.g. 6-ACA, another amino acid, 5-FVA, succinic acid/succinate, acetic acid/acetate, these terms are meant to include the protonated carboxylic acid (free acid), the corresponding carboxylate (its conjugated base) as well as a salt thereof, unless specified otherwise. Likewise, when referring to an amine, this is meant to include the protonated amine (typically cationic, e.g. R—NH₃⁺) and the unprotonated amine (typically uncharged, e.g. R—NH₂). When referring herein to amino acids, e.g. 6-ACA, this term is meant to include amino acids in their zwitterionic form (in which the amino group is in the protonated and the carboxylate group is in the deprotonated form), the amino acid in which the amino group is protonated and the carboxylic group is in its neutral form, and the amino acid in which the amino group is in its neutral form and the carboxylate group is in the deprotonated form, as well as salts thereof.

When referring to a compound of which several isomers exist (e.g. a cis and a trans isomer, an R and an S enantiomer), the compound in principle includes all enantiomers, diastereomers and cis/trans isomers of that compound that may be used in the particular method of the invention.

When an enzyme is mentioned with reference to an enzyme class (EC) between brackets, the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at http://www.chem.qmul.ac.uk/iubmb/enzyme/. Other suitable enzymes that have not (yet) been classified in a specified class but may be classified as such, are meant to be included.

If referred herein to a protein or gene by reference to a accession number, this number in particular is used to refer to a protein or gene having a sequence as found in Uniprot on 11 Mar. 2008, unless specified otherwise.

As used herein, the term “functional analogue” of a nucleic acid at least includes other sequences encoding an enzyme having the same amino acid sequence and other sequences encoding a homologue of such enzyme.

The term “homologue” is used herein in particular for polynucleotides or polypeptides having a sequence identity of at least 30%, preferably at least 40%, more preferably at least 60%, more preferably at least 65%, more preferably at least 70%, more preferably at least 75%, more preferably at least 80%, in particular at least 85%, more in particular at least 90%, at least 91%, at least 92%, at least 93%, at least 94%, at least 95%, at least 96%, at least 97%, at least 98% or at least 99%. The term homologue is also meant to include nucleic acid sequences (polynucleotide sequences) which differ from another nucleic acid sequence due to the degeneracy of the genetic code and encode the same polypeptide sequence.

Sequence identity or similarity is herein defined as a relationship between two or more polypeptide sequences or two or more nucleic acid sequences, as determined by comparing the sequences. Usually, sequence identities or similarities are compared over the whole length of the sequences, but may however also be compared only for a part of the sequences aligning with each other. In the art, “identity” or “similarity” also means the degree of sequence relatedness between polypeptide sequences or nucleic acid sequences, as the case may be, as determined by the match between such sequences. Preferred methods to determine identity or similarity are designed to give the largest match between the sequences tested. In context of this invention a preferred computer program method to determine identity and similarity between two sequences includes BLASTP and BLASTN (Altschul, S. F. et al., J. Mol. Biol. 1990, 215, 403-410, publicly available from NCBI and other sources (BLAST Manual, Altschul, S., et al., NCBI NLM NIH Bethesda, Md. 20894). Preferred parameters for polypeptide sequence comparison using BLASTP are gap open 10.0, gap extend 0.5, Blosum 62 matrix. Preferred parameters for nucleic acid sequence comparison using BLASTN are gap open 10.0, gap extend 0.5, DNA full matrix (DNA identity matrix).

A heterologous biocatalyst, in particular a heterologous cell, as used herein, is a biocatalyst comprising a heterologous protein or a heterologous nucleic acid (usually as part of the cell's DNA or RNA) The term “heterologous” when used with respect to a nucleic acid sequence (DNA or RNA), or a protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. It is understood that heterologous DNA in a heterologous organism is part of the genome of that heterologous organism. Heterologous nucleic acids or proteins are not endogenous to the cell into which they are introduced, but have been obtained from another cell or synthetically or recombinantly produced. Generally, though not necessarily, such nucleic acids encode proteins that are not normally produced by the cell in which the DNA is transcribed or expressed. Similarly heterologous RNA encodes for proteins not normally expressed in the cell in which the heterologous RNA is present. Heterologous nucleic acids and proteins may also be referred to as foreign nucleic acids or proteins. Any nucleic acid or protein that one of skill in the art would recognise as heterologous or foreign to the cell in which it is expressed is herein encompassed by the term heterologous nucleic acid or protein.

When referred to an enzyme or another biocatalytic moiety from a particular source, recombinant enzymes or other recombinant biocatalytic moieties, originating from a first organism, but actually produced in a (genetically modified) second organism, are specifically meant to be included as enzymes or other biocatalytic moieties, from that first organism.

In a method of the invention, a biocatalyst is used, i.e. at least one reaction step in the method is catalysed by a biological material or moiety derived from a biological source, for instance an organism or a biomolecule derived there from. The biocatalyst may in particular comprise one or more enzymes. A biocatalytic reaction may comprise one or more chemical conversions of which at least one is catalyzed by a biocatalyst. Thus the ‘biocatalyst’ may accelerate a chemical reaction in at least one reaction step in the preparation of AKP from AKG, at least one reaction step in the preparation of 5-FVA or AAP from AKP, at least one reaction step in the preparation of 6-ACA from 5-FVA, at least one reaction step in the preparation of 6-ACA from AAP or at least one reaction step in the preparation of caprolactam from 6-ACA.

The biocatalyst may be used in any form. In an embodiment, one or more enzymes form part of a living organism (such as living whole cells). The enzymes may perform a catalytic function inside the cell. It is also possible that the enzyme may be secreted into a medium, wherein the cells are present. In an embodiment, one or more enzymes are used isolated from the natural environment (isolated from the organism it has been produced in), for instance as a solution, an emulsion, a dispersion, (a suspension of) freeze-dried cells, a lysate, or immobilised on a support. The use of an enzyme isolated from the organism it originates from may in particular be useful in view of an increased flexibility in adjusting the reaction conditions such that the reaction equilibrium is shifted to the desired side.

Living cells may be growing cells, resting or dormant cells (e.g. spores) or cells in a stationary phase. It is also possible to use an enzyme forming part of a permeabilized cell (i.e. made permeable to a substrate for the enzyme or a precursor for a substrate for the enzyme or enzymes).

The biocatalyst (used in a method of the invention) may in principle be any organism, or be obtained or derived from any organism. This organism may be a naturally occurring organism or a heterologous organism. The heterologous organism is typically a host cell which comprises at least one nucleic acid sequence encoding a heterologous enzyme, capable of catalysing at least one reaction step in a method of the invention. The organism from which the heterologous nucleic acid sequence originates may be eukaryotic or prokaryotic. In particular said organisms may be independently selected from animals (including humans), plants, bacteria, archaea, yeasts and fungi.

The host cell may be eukaryotic or prokaryotic. In an embodiment, the host cell is selected from the group of fungi, yeasts, euglenoids, archaea and bacteria. The host cell may in particular be selected from the group of genera consisting of Aspergillus, Penicillium, Ustilago, Cephalosporium, Trichophytum, Paecilomyces, Pichia, Hansenula, Saccharomyces, Candida, Kluyveromyces, Yarrowia, Bacillus, Corynebacterium, Escherichia, Azotobacter, Frankia, Rhizobium, Bradyrhizobium, Anabaena, Synechocystis, Microcystis, Klebsiella, Rhodobacter, Pseudomonas, Thermus, Deinococcus Gluconobacter, Methanosphaera, Methanobrevibacter, Methanospirillum, Methanococcus, Methanobacterium, Methanocaldococcus and Methanosarcina. In particular, the host strain and, thus, host cell for use in a method of the invention may be selected from the group of Escherichia coli, Azotobacter vinelandii, Klebsiella pneumoniae, Anabaena sp., Synechocystis sp., Microcystis aeruginosa, Deinococcus radiourans, Deinococcus geothermalis, Thermus thermophilus, Bacillus subtilis, Bacillus amyloliquefaciens, Bacillus methanolicus, Corynebacterium glutamicum, Aspergillus niger, Penicillium chrysogenum, Penicillium notatum, Paecilomyces carneus, Cephalosporium acremonium, Ustilago maydis, Pichia pastoris, Saccharomyces cerevisiae, Kluyveromyces lactis, Candida maltosa, Yarrowia lipolytica, Hansenula polymorpha, Sulfolobus solfataricus, Methanobacterium thermoautothrophicum, Methanococcus maripaludis, Methanocaldococcus jannashii, Methanosphaera stadtmanae, Methanococcus voltae, Methanosarcina acetivorans, Methanosarcina barkeri, Methanosarcina acetivorans, Methanospirillum hungatei, Methanosaeta thermophila, Methanobrevibacter smithii, Methanococcus vannielii, Methanococcus aeolicus and Methanosarcina mazei host cells

In particular in an embodiment wherein AKP is to be converted into a further product, for instance 5-FVA, AAP, diaminohexane or 6-ACA, it is considered advantageous that the host cell is an organism naturally capable of converting AKP to such product or at least capable of catalysing at least one of the necessary reactions. For instance, Escherichia coli has aminotransferase activity, whereby E. coli may catalyse the formation of AAP from AKP (see also below) or the conversion of 5-FVA (which may be formed in the cell if the cell also contains a suitable decarboxylase, see also below) to 6-ACA.

Advantageously, the host cell is an organism comprising a biocatalyst catalysing the amino adipate pathway for lysine biosynthesis (also termed AAA pathway) or a part thereof (such as lower eukaryotes: fungi, yeasts, euglenoids; certain bacteria, e.g. Thermus, Deinococcus; Archaea) or comprising a biocatalyst for nitrogen fixation via a nitrogenase.

In a preferred embodiment, the host cell is an organism with a high flux through the AAA pathway, such as Penicillium chrysogenum, Ustilago maydis or an organism adapted, preferably optimised, for lysine production. A high flux is defined as at least 20%, more preferred at least 50%, even more preferred at least 70%, most preferred at least 100% of the rate required to supply lysine for biosynthesis of cellular protein in the respective organism under the chosen production conditions.

In a preferred embodiment, the host cell is an organism with high levels of homocitrate being produced, which may be a naturally occurring or a heterologous organism. Such an organism may be obtained by expressing a homocitrate synthase required for formation of the essential cofactor found in nitrogenases or a homologue thereof.

In an embodiment, the host cell comprises a heterologous nucleic acid sequence originating from an animal, in particular from a part thereof—e.g. liver, pancreas, brain, kidney, heart or other organ. The animal may in particular be selected from the group of mammals, more in particular selected from the group of Leporidae, Muridae, Suidae and Bovidae.

In an embodiment, the host cell comprises a heterologous nucleic acid sequence originating from a plant. Suitable plants in particular include plants selected from the group of Asplenium; Cucurbitaceae, in particular Curcurbita, e.g. Curcurbita moschata (squash), or Cucumis; Brassicaceae, in particular Arabidopsis, e.g. A. thaliana; Mercurialis, e.g. Mercurialis perennis; Hydnocarpus; and Ceratonia.

In an embodiment, the host cell comprises a heterologous nucleic acid sequence originating from a bacterium. Suitable bacteria may in particular be selected amongst the group of Vibrio, Pseudomonas, Bacillus, Corynebacterium, Brevibacterium, Enterococcus, Streptococcus, Actinomycetales, Klebsiella, Lactococcus, Lactobacillus, Clostridium, Escherichia, Klebsiella, Anabaena, Microcystis, Synechocystis, Rhizobium, Bradyrhizobium, Thermus, Mycobacterium, Zymomonas, Proteus, Agrobacterium, Geobacillus, Acinetobacter, Azotobacter, Ralstonia, Rhodobacter, Paracoccus, Novosphingobium, Nitrosomonas, Legionella, Neisseria, Rhodopseudomonas, Staphylococcus, Deinococcus and Salmonella.

In an embodiment, the host cell comprises a heterologous nucleic acid sequence originating from an archaea. Suitable archaea may in particular be selected amongst the group of Archaeoglobus, Aeropyrum, Halobacterium, Methanosarcina, Methanococcus, Thermoplasma, Thermococcus, Pyrobaculum, Methanospirillum, Pyrococcus, Sulfolobus, Methanococcus, Methanosphaera, Methanopyrus, Methanobrevibacter, Methanocaldococcus and Methanobacterium.

In an embodiment, the host cell comprises a heterologous nucleic acid sequence originating from a fungus. Suitable fungi may in particular be selected amongst the group of Rhizopus, Phanerochaete, Emericella, Ustilago, Neurospora, Penicillium, Cephalosporium, Paecilomyces, Trichophytum and Aspergillus.

In an embodiment, the host cell comprises a heterologous nucleic acid sequence originating from a yeast. A suitable yeast may in particular be selected amongst the group of Candida, Hansenula, Kluyveromyces, Yarrowia, Schizosaccharomyces, Pichia, Yarrowia and Saccharomyces.

It will be clear to the person skilled in the art that use can be made of a biocatalyst wherein a naturally occurring biocatalytic moiety (such as an enzyme) is expressed (wild type) or a mutant of a naturally occurring biocatalytic moiety with suitable activity in a method according to the invention. Properties of a naturally occurring biocatalytic moiety may be improved by biological techniques known to the skilled person, e.g. by molecular evolution or rational design. Mutants of wild-type biocatalytic moieties can for example be made by modifying the encoding DNA of an organism capable of producing a biocatalytic moiety (such as an enzyme) using mutagenesis techniques known to the person skilled in the art. These include random mutagenesis, site-directed mutagenesis, directed evolution, and gene recombination. In particular the DNA may be modified such that it encodes an enzyme that differs by at least one amino acid from the wild-type enzyme, so that it encodes an enzyme that comprises one or more amino acid substitutions, deletions and/or insertions compared to the wild-type, or such that the mutants combine sequences of two or more parent enzymes or by effecting the expression of the thus modified DNA in a suitable (host) cell. The latter may be achieved by methods known to the skilled person such as codon optimisation or codon pair optimisation, e.g. based on a method as described in WO 2008/000632.

A mutant biocatalyst may have improved properties, for instance with respect to one or more of the following aspects: selectivity towards the substrate, activity, stability, solvent tolerance, pH profile, temperature profile, substrate profile, susceptibility to inhibition, cofactor utilisation and substrate-affinity. Mutants with improved properties can be identified by applying e.g. suitable high through-put screening or selection methods based on such methods known to the skilled person in the art.

In accordance with the invention, AKP is prepared from AKG. The AKG may in principle be obtained in any way. In particular, AKG may be obtained biocatalytically by providing the heterologous biocatalyst with a suitable carbon source that can be converted into AKG, for instance by fermentation of the carbon source. In an advantageous method AKG is prepared making use of a whole cell biotransformation of the carbon source to form AKG.

The carbon source may in particular contain at least one compound selected from the group of monohydric alcohols, polyhydric alcohols, carboxylic acids, carbon dioxide, fatty acids, glycerides, including mixtures comprising any of said compounds. Suitable monohydric alcohols include methanol and ethanol, Suitable polyols include glycerol and carbohydrates. Suitable fatty acids or glycerides may in particular be provided in the form of an edible oil, preferably of plant origin.

In particular a carbohydrate may be used, because usually carbohydrates can be obtained in large amounts from a biologically renewable source, such as an agricultural product, preferably an agricultural waste-material. Preferably a carbohydrate is used selected from the group of glucose, fructose, sucrose, lactose, saccharose, starch, cellulose and hemi-cellulose. Particularly preferred are glucose, oligosaccharides comprising glucose and polysaccharides comprising glucose.

In an embodiment of the invention AKG is converted into AKA using a biocatalyst for the conversion of AKG into AKA, part of said biocatalyst originating from the AAA pathway for lysine biosynthesis. Such conversion may involve a single or a plurality of reaction steps, which steps may be catalysed by one or more biocatalysts.

The biocatalyst for catalysing the conversion of AKG into AKA or parts thereof may be homologous or heterologous. In particular, the biocatalyst forming part of the AAA pathway for lysine biosynthesis may be found in an organism selected from the group of yeasts, fungi, archaea and bacteria, in particular from the group of Penicillium, Cephalosporium, Paecilomyces, Trichophytum, Aspergillus, Phanerochaete, Emericella, Ustilago, Schizosaccharomyces, Saccharomyces, Candida, Kluyveromyces, Yarrowia, Pichia, Hansenula, Thermus, Deinococcus, Pyrococcus, Sulfolobus, Thermococcus, Methanococcus, Methanosarcina, Methanocaldococcus, Methanosphaera, Methanopyrus, Methanobrevibacter, Methanospirillum and Methanothermobacter. A suitable biocatalyst may be found in an organism able to produce homocitrate,e.g. a biocatalyst for the nitrogenase complex in nitrogen fixing bacteria such as cyanobacteria (e.g. Anabaena, Microcystis, Synechocystis) Rhizobiales (e.g. Rhizobium, Bradyrhizobium), γ-proteobacteria (e.g. Pseudomonas, Azotobacter, Klebsiella) and actinobacteria (e.g. Frankia). Thus, if a biocatalyst is used based on a host cell naturally comprising the AAA pathway for lysine biosynthesis or parts thereof, this system may be homologous.

In a preferred embodiment of the invention a high productivity of AKA by the biocatalyst is desired. A biocatalyst containing the AAA pathway for lysine biosynthesis or parts thereof may be modified by methods known in the art such as mutation/screening or metabolic engineering to this effect. A high level of AKA can be generated by increasing the activity of enzymes involved in its formation and/or decreasing the activity involved in its conversion to e.g. amino adipate.

Enzymes involved in formation of AKA include homocitrate synthase (EC 2.3.3.14), homo aconitase (EC 4.2.1.36), and homoisocitrate dehydrogenase (EC 1.1.1.87). The activity for these enzymes in the host cell can be increased by methods known in the art such as (over-) expression of genes encoding the respective enzyme and/or functional homologues, alleviating inhibitions by substrates, products or other compounds, or improving catalytic properties of the enzymes by molecular evolution or rational design. A preferred method to perform directed evolution may be based on WO 2003/010183.

As it is undesired that the AKA that is produced is converted to aminoadipate (AAA)—which would be a further step in the pathway for lysine biosynthesis—it is preferred that the heterologous biocatalyst has low or no activity of an enzyme catalysing this conversion, in particular an aminotransferase, such as aminoadipate aminotransferase (EC 2.6.1.39) or amino acid dehydrogenase capable of catalysing this conversion. Thus, in case the host cell providing the biocatalyst comprises a gene encoding such an enzyme, such gene is preferably inactivated, knocked out, or the expression of such gene is reduced. As this step is essential in the AAA pathway for lysine production a host cell which has limited, minimal activity to supply the required amount of lysine for growth and maintenance but is not capable of high level conversions of AKA to AAA is advantageous. In particular in case Penicillium chrysogenum is the host, the aminotransferase may have the sequence of Sequence ID 68, or a homologue thereof.

Inactivation of a gene encoding an undesired activity may be accomplished, by several methods. One approach is a temporary one using an anti-sense molecule or RNAi molecule (e.g. based on Kamath et al. 2003. Nature 421:231-237). Another is using a regulatable promoter system, which can be switched off using external triggers like tetracycline (e.g. based on Park and Morschhauser, 2005, Eukaryot. Cell. 4:1328-1342). Yet another one is to apply a chemical inhibitor or a protein inhibitor or a physical inhibitor (e.g. based on Tour et al. 2003. Nat Biotech 21:1505-1508). A much preferred method is to remove the complete gene(s) or a part thereof, encoding the undesired activity. To obtain such a mutant one can apply state of the art methods like Single Cross-Over Recombination or Double Homologous Recombination. For this, one needs to construct an integrative cloning vector that may integrate at the predetermined target locus in the chromosome of the host cell. In a preferred embodiment of the invention, the integrative cloning vector comprises a DNA fragment, which is homologous to a DNA sequence in a predetermined target locus in the genome of host cell for targeting the integration of the cloning vector to this predetermined locus. In order to promote targeted integration, the cloning vector is preferably linearized prior to transformation of the host cell. Linearization is preferably performed such that at least one but preferably either end of the cloning vector is flanked by sequences homologous to the target locus. The length of the homologous sequences flanking the target locus is preferably at least 0.1 kb, even preferably at least 0.2 kb, more preferably at least 0.5 kb, even more preferably at least 1 kb, most preferably at least 2 kb. The length that finally is best suitable in an experiment depends on the organism, the sequence and length of the target DNA.

The efficiency of targeted integration of a nucleic acid construct into the genome of the host cell by homologous recombination, i.e. integration in a predetermined target locus, is preferably increased by augmented homologous recombination abilities of the host cell. Such phenotype of the cell preferably involves a deficient hdfA or hdfB gene as described in WO 05/95624. WO 05/95624 discloses a preferred method to obtain a filamentous fungal cell comprising increased efficiency of targeted integration by preventing non-homologous random integration of DNA fragments into the genome. The vector system may be a single vector or plasmid or two or more vectors or plasmids, which together contain the total DNA to be introduced into the genome of the host cell.

Fungal cells may be transformed by protoplast formation, protoplast transformation, and regeneration of the cell wall. Suitable procedures for transformation of fungal host cells are described in EP 238023 and Yelton et al. (1984. Proc. Nat. Acad. Sci. USA 81:1470-1474). Suitable procedures for transformation of filamentous fungal host cells using Agrobacterium tumefaciens are described by de Groot M. J. et al. (1998. Nat. Biotechnol. 16:839-842. Erratum in: Nat. Biotechnol. 1998. 16:1074). Other methods like electroporation, described for Neurospora crassa, may also be applied.

Fungal cells are transfected using co-transformation, i.e. along with gene(s) of interest also a selectable marker gene is transformed. This can be either physically linked to the gene of interest (i.e. on a plasmid) or on a separate fragment. Following transfection transformants are screened for the presence of this selection marker gene and subsequently analyzed for the integration at the preferred predetermined genomic locus. A selectable marker is a product, which provides resistance against a biocide or virus, resistance to heavy metals, prototrophy to auxotrophs and the like. Useful selectable markers include, but are not limited to, amdS (acetamidase), argB (ornithinecarbamoyltransferase), bar (phosphinothricinacetyl-transferase), hygB (hygromycin phosphotransferase), niaD (nitrate reductase), pyrG (orotidine-5′-phosphate decarboxylase), sC or sutB (sulfate adenyltransferase), trpC (anthranilate synthase), ble (phleomycin resistance protein), as well as equivalents thereof. The most preferred situation is providing a DNA molecule comprising a first DNA fragment comprising a desired replacement sequence (i.e. the selection marker gene) flanked at its 5′ and 3′ sides by DNA sequences substantially homologous to sequences of the chromosomal DNA flanking the target sequence. Cells wherein the target sequence in the chromosomal DNA sequence is replaced by the desired replacement sequence can be selected by the presence of the selectable marker of the first DNA fragment. To increase the relative frequency of selecting the correct mutant microbial strain, a second DNA fragment comprising an expression cassette comprising a gene encoding a selection marker and regulatory sequences functional in the eukaryotic cell can be operably linked to the above described fragment (i.e. 5′-flank of target locus+selection marker gene+3′-flank of target locus) and cells wherein the target sequence in the chromosomal DNA sequence is replaced by the desired replacement sequence can be selected by the presence of the selectable marker of the first DNA fragment and the absence of the second selection marker gene.

In case the enzyme system forming part of the aminoadipate pathway for lysine biosynthesis is heterologous to the host cell, it is preferred that no genes are included into the host cell that encode an enzyme catalysing the conversion of ketoadipate into aminoadipate. The term ‘enzyme system’ is in particular used herein for a single enzyme or a group of enzymes whereby a specific conversion can be catalysed. Said conversion may comprise one or more chemical reactions with known or unknown intermediates e.g. the conversion of AKG into AKA or the conversion of AKA into AKP. Such system may be present inside a cell or isolated from a cell. It is known that aminotransferases often have a wide substrate range. It may be desired to decrease activity of one or more such enzymes present in a host cell such that activity in the conversion of AKA to AAA is reduced, whilst maintaining relevant catalytic functions for biosynthesis of other amino acids or cellular components. Also a host cell devoid of any other enzymatic activity resulting in the conversion of AKA to an undesired side product is preferred.

In a further embodiment, AKG is converted into AKA, making use of at least one heterologous biocatalyst catalysing the C₁-elongation of AKG into AKA. One or more biocatalysts may be used. Said biocatalyst or biocatalysts may comprise one or enzymes originating from one or more source organisms (e.g. comprise more than one enzyme originating from different source organisms). A suitable biocatalyst for preparing AKA from AKG may in particular be selected amongst biocatalysts catalysing C₁-elongation of alpha-ketoglutaric acid into alpha-ketoadipic acid and/or C₁-elongation of alpha-ketoadipic acid into alpha-ketopimelic acid.

AKA prepared from AKG may thereafter be converted into AKP, making use of at least one heterologous biocatalyst catalysing the elongation of AKA into AKP. These biocatalysts may be the same as or different from the biocatalysts catalysing the conversion of AKG into AKA by C₁-elongation. One or more than one biocatalyst may be used for conversion of AKA to AKP. Said biocatalyst(s) may comprise one or more enzymes originating from one or more source organisms (e.g. comprise more than one enzyme originating from different source organisms).

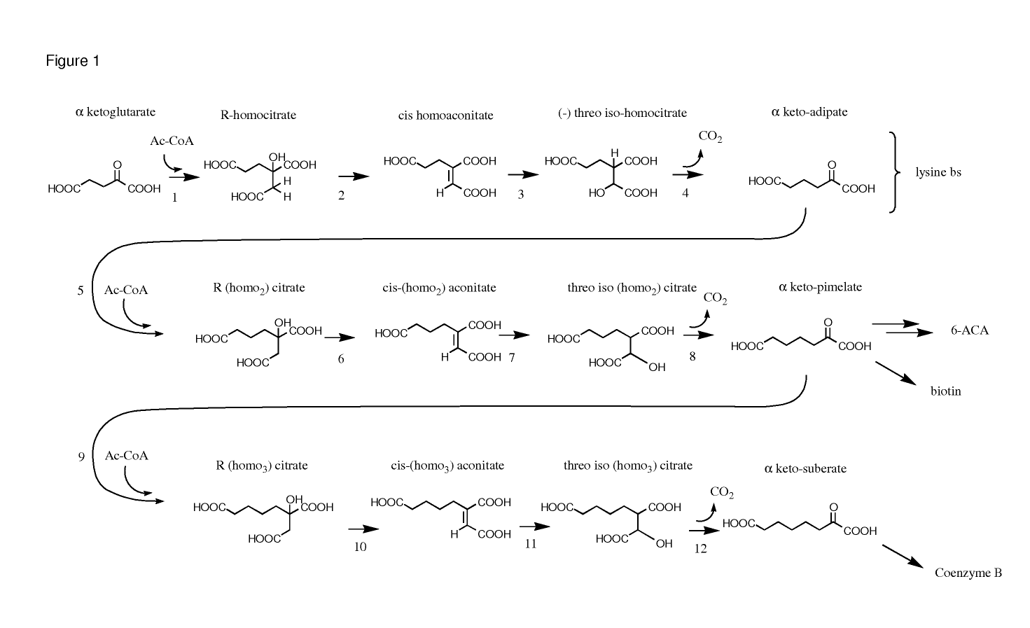

A biosynthetic pathway making use of C₁-elongation is known to exist in methanogenic Archaea as part of coenzyme B biosynthesis and part of biotin biosynthesis. Coenzyme B is considered essential for methanogenesis in these organisms and alpha-ketosuberate is an important intermediate in coenzyme B biosynthesis. In such methanogenic Archaea alpha-ketoglutaric acid is converted to alpha-ketoadipic acid, then alpha-ketopimelic acid and finally alpha-ketosuberic acid by successive addition of methylene groups following a plurality of reaction steps (see also FIG. 1):

a. alpha-keto-acid of length Cₙ+acetyl-CoA→homoₙcitrate+CoA-SH (steps 1, 5 and 9 in FIG. 1)
b. homoₙ-citrate← →homoₙ-aconitate (catalyzed by homoₙ-citrate dehydratase (steps 2, 6 and 10 in FIG. 1)
c. homoₙaconitate← →isohomoₙ-citrate (steps 3, 7 and 11) in FIG. 1)
d. homoₙ-isocitrate+NADP⁺→alpha-keto-acid of length Cₙ₊₁+NADPH+H⁺+CO₂ (steps 4, 8 and 12 in FIG. 1)
wherein n is selected from 1-4.

This repetitive reaction sequence has been described for the methanogens Methanosarcina thermophila and Methanocaldococcus jannashii. Similar non-iterative reactions are involved in C₁-extension of other α-ketocarboxylic acids in other metabolic pathways such as the conversion of oxaloacetate to α-ketoglutarate in the oxidative citrate cycle, conversion of alpha-isovalerate to α-isocaproate as part in the isopropylmalate pathway to leucine, conversion of alpha-ketoglutarate to α-ketoadipate in the AAA pathway to lysine, conversion of pyruvate to alpha-ketobutyrate in the pyruvate pathway to isoleucine, and in the conversion of maleate to pyruvate. Collectively these reactions are defined as “C₁-elongation”.

Several genes and enzymes involved in C₁-elongations have been described and characterised from M. jannashii. It was shown that these enzymes and the encoding genes are similar to each other and to other enzymes and their encoding genes involved in C₁-elongations in other organisms. A subset of enzymes for the iterative elongation of alpha-ketoglutarate to alpha-ketosuberate via alpha-ketoadipate and alpha-ketopimelate has been characterised biochemically and was called “Aks”. Some of the genes encoding these enzymes have been identified in the genome sequence of M. jannashii and others have been proposed.

The inventors have realised that C₁-elongation can be used to prepare AKA or AKP on an industrial scale, such that AKA or AKP can be made available as an intermediate for the preparation of special compounds or commodity products, such as diaminohexane or caprolactam, by incorporating one or more nucleic acid sequences encoding an enzyme system involved in C₁ elongation into a suitable host cell.

The enzyme system for catalysing C₁ elongation thereby forming AKA or AKP may in particular comprise one or more enzymes selected from the group of homoₙ-citrate synthases, homoₙ-aconitases and iso-homoₙ-citrate dehydrogenases, wherein n is selected from 1-4.

A homoₙ-citrate synthase may in particular catalyse “reaction a” of the C₁-elongation. A homoₙ-citrate synthase is defined as an enzyme capable of condensing an alpha-keto carboxylic diacid of chain length C₄₊ₙ with acetyl-CoA resulting in formation of homoₙ-citrate wherein n is selected from 1-4. The homoₙ-citrate synthase may in particular be an enzyme that is or can be classified in EC 2.3.3. More in particular, a suitable homoₙ-citrate synthase may be selected amongst homocitrate synthases (EC 2.3.3.14), or may be classified in EC 2.3.3.1, 2.3.3.2, 2.3.3.4 or 2.3.3.9. Particularly preferred is AksA or a homologue thereof having homo₍ₙ₎citrate activity.

A homoₙ-aconitase may in particular catalyse “reaction b” and/or “reaction c” of the C₁-elongation. A homoₙ-aconitase is defined as an enzyme capable of converting homoₙ-citrate to iso-homoₙ-citrate via a homoₙ-aconitate intermediate or at least one of the reversible half reactions (i.e. homoₙ-aconitate to homoₙ-citrate or homoₙ-aconitate to iso-homoₙ-citrate) wherein n is selected from 1-4. The homoₙ-aconitase may in particular be an enzyme that is or can be classified in EC 4.2.1. More in particular, a suitable homoₙ-aconitase may be selected amongst homoaconitase (EC 4.2.1.36), or may be classified in EC 4.2.1.3, 4.2.1.33, 4.2.1.79 and 4.2.1.99. Particularly preferred is an enzyme selected from the group of AksD, AksE, homologues of AksD and homologues of AksE having homoₙ-aconitase activity.

A homoₙ-isocitrate dehydrogenase may in particular catalyse “reaction d” of the C₁-elongation. A iso-homoₙ-citrate dehydrogenase is defined as an enzyme capable of converting iso-homoₙ-citrate to an α-keto-carboxylic-diacid of chain length C₅₊ₙ wherein n is selected from 1-4 and thereby releasing CO₂. The iso-homoₙ-citrate dehydrogenase may in particular be an enzyme that is or can be classified in EC 1.1.1. More in particular, a suitable iso-homoₙ-citrate dehydrogenase may be selected amongst iso-homocitrate dehydrogenase (EC 1.1.1.87), or may be classified in EC 1.1.136, 1.1.137, 1.1.1.38, 1.1.139, 1.1.1.40, 1.1.1.41, 1.1.1.42, 1.1.1.82, 1.1.1.83, 1.1.1.84, 1.1.1.85 and 1.1.1.286. Particularly preferred is AksF or a homologue thereof having homoₙ-isocitrate dehydrogenase activity.

Methanogens may serve as biocatalysts for production of AKP or can be used as a source for such biocatalysts. Suitable biocatalysts may be identified by searching for protein and nucleotide sequences similar to known enzymes from C₁-elongations pathways. Similar sequences can efficiently be identified in sequence databases using bioinformatic techniques well known in the art. Molecular biology methods known in the art such as Southern hybridization or PCR techniques employing degenerate oligonucleotides can be used to identify similar genes in cultured organisms and environmental samples. After cloning and sequencing such biocatalysts may be utilized for AKP production in a heterologous host.

In particular, one or more enzymes for catalysing C₁ elongation may be used from a methanogen selected from the group of Methanococcus, Methanospirillum, Methanocaldococcus, Methanosarcina, Methanothermobacter, Methanosphaera, Methanopyrus and Methanobrevibacter. More specifically one or more enzymes may be used from a methanogen selected from the group of Methanothermobacter thermoautotropicum, Methanococcus maripaludis, Methanosphaera stadtmanae, Methanopyrus kandleri, Methanosarcina thermophila, Methanobrevibacter smithii, Methanococcus vannielii, Methanospirillum hungatei, Methanosaeta thermophila Methanosarcina acetivorans and Methanococcus aeolicus.

Further, suitable enzymes for catalysing C₁ elongation of AKG and/or AKA may e.g. be found in organisms comprising an enzyme system for catalysing lysine biosynthesis via the aminoadipate pathway or parts thereof or contain homologues thereof as part of other metabolism such as e.g. homocitrate synthase involved in nitrogen fixation. In particular organisms selected from the group of yeasts and fungi, such as Penicillium, Cephalosporium, Aspergillus, Phanerochaete, Emericella, Ustilago, Paecilomyces, Trichophytum, Yarrowia, Hansenula, Schizosaccharomyces, Saccharomyces, Candida, Kluyveromyces, in particular Penicillium chrysogenum, Penicillium notatum, Paecilomyces carneus, Paecilomyces persinicus, Cephalosporium acremonium, Aspergillus niger, Emericella nidulans, Aspergillys oryzae, Ustilago maydis, Schizosaccharomyces pombe, Saccharomyces cerevisiae, Yarrowia lipolytica, Hansenula polymorpha, Candida albicans, Candida maltosa, and Kluyveromyces lactis; bacteria, such as Azotobacter, Pseudomonas, Klebsiella, Deinococcus, Thermus, in particular Azotobacter vinelandii, Pseudomonas stutzerii, Klebsiella pneumoniae, Deinococcus radiourans, Deinococcus geothermalis, Thermus thermophilus; and archae, such as Pyrococcus, Sulfolobus, Thermococcus, Methanococcus, Methanocaldococcus, Methanosphaera, Methanopyrus, Methanospirillum, Methanobrevibacter, Methanosarcina and Methanothermobacter, in particular Pyrococcus horikoshii, Sulfolobus solfataricus, Thermococcus kodakarensis, Methanococcus maripaludis, Methanococcus aeolicus, Methanococcus vannielii, Methanoca/dococcus jannashii, Methanosphaera stadtmanae, Methanopyrus kandleri, Methanobrevibacter smithii, Methanosarcina thermophilus, Methanospirillum hungatei, Methanosaeta thermophila, Methanosarcina acetivorans and Methanothermobacter thermoautotrophicum. Such yeast, fungus, bacterium, archaeon or other organism may in particular provide a homocitrate synthase capable of catalysing “reaction a” in the elongation of AKG to AKA and optionally the elongation of AKA to APK.

Further, suitable biocatalysts for catalysing a reaction step in the preparation of AKP may be found in Asplenium or Hydnocarpus, in particular Asplenium septentrionale or Hydnocarpus anthelminthica, which naturally are capable of producing AKP.

In a preferred method one or more enzymes selected from the group of Aks enzymes and homologues thereof, in particular from the group of AksA, AksD, AksE, AksF and homologues thereof are used. Examples of homologues for these Aks enzymes and the genes encoding these enzymes are given in the Tables on the following pages.

| | Enzyme | | | |
|---|---|---|---|---|
| Step | name | Organism | gene | Protein |
| 1 | AksA | Methanocaldococcus jannashii | MJ0503 | NP_247479 |
| | | Methanothermobacter thermoautotropicum ΔH | MTH1630 | NP_276742 |
| | | Methanococcus maripaludis S2 | MMP0153 | NP_987273 |
| | | Methanococcus maripaludis C5 | MmarC5_1522 | YP_001098033 |
| | | Methanococcus maripaludis C7 | MmarC7_1153 | YP_001330370 |
| | | Methanospaera stadtmanae DSM 3091 | Msp_0199 | YP_447259 |
| | | Methanopyrus kandleri AV19 | MK1209 | NP_614492 |
| | | Methanobrevibacter smithii ATCC35061 | Msm_0722 | YP_001273295 |
| | | Methanococcus vannielii SB | Mevan_1158 | YP_001323668 |
| | | Klebsiella pneumoniae | nifV | P05345 |
| | | Azotobacter vinelandii | nifV | P05342 |
| | | Pseudomonas stutzerii | nifV | ABP79047 |
| | | Methanococcus aeolicus Nankai 3 | Maeo_0994 | YP_001325184 |
| 2, 3 | AksD | Methanocaldococcus jannashii | MJ1003 | NP_247997 |
| | | Methanothermobacter thermoautotropicum ΔH | MTH1386 | NP_276502 |
| | | Methanococcus maripaludis S2 | Mmp1480 | NP_988600 |
| | | Methanococcus maripaludis C5 | MmarC5_0098 | YP_001096630 |
| | | Methanococcus maripaludis C7 | MmarC7_0724 | YP_001329942 |
| | | Methanospaera stadtmanae DSM 3091 | Msp_1486 | YP_448499 |
| | | Methanopyrus kandleri AV19 | MK1440 | NP_614723 |
| | | Methanobrevibacter smithii ATCC35061 | Msm_0723 | YP_001273296 |
| | | Methanococcus vannielii SB | Mevan_0789 | YP_001323307 |
| | | Methanococcus aeolicus Nankai 3 | Maeo_0311 | YP_001324511 |
| | | Methanosarcina acetivorans | MA3085* | NP_617978* |
| | | Methanospirillum hungatei JF-1 | Mhun_1800* | YP_503240* |
| | | Methanosaeta thermophila PT | Mthe_0788* | YP_843217* |
| | | Methanosphaera stadtmanae DSM 3091 | Msp_1100* | YP_448126* |

References to gene and protein can be found via www.ncbi.nlm.nih.gov/(for listed gene/protein marked with an *: as available on 2 Mar. 2010, for the others: as available on 15 Apr. 2008).

| | Enzyme | | | |
|---|---|---|---|---|
| Step | name | Orgamism | gene | Protein |
| 2, 3 | AksE | Methanocaldococcus jannashii | MJ1271 | NP_248267 |
| | | Methanothermobacter thermoautotropicum ΔH | MTH1387 | NP_276503 |
| | | Methanococcus maripaludis S2 | MMP0381 | NP_987501 |
| | | Methanococcus maripaludis C5 | MmarC5_1257 | YP_001097769 |
| | | Methanococcus maripaludis C7 | MmarC7_1379 | YP_001330593 |
| | | Methanospaera stadtmanae DSM 3091 | Msp_1485 | YP_448498 |
| | | Methanopyrus kandleri AV19 | MK0781 | NP_614065 |
| | | Methanobrevibacter smithii ATCC35061 | Msm_0847 | YP_001273420 |
| | | Methanococcus vannielii SB | Mevan_1368 | YP_001323877 |
| | | Methanococcus aeolicus Nankai 3 | Maeo_0652 | YP_001324848 |
| | | Methanosarcina acetivorans | MA3751* | NP_618624* |
| | | Methanospirillum hungatei JF-1 | Mhun_1799* | YP_503239* |
| | | Methanosphaera stadtmanae DSM 3091 | Msp_0374* | YP_447420* |
| | | Methanosaeta thermophila PT | Mthe_0853* | YP_843282* |
| 4 | AksF | Methanocaldococcus jannashii | MJ1596 | NP_248605 |
| | | Methanothermobacter thermoautotropicum ΔH | MTH184 | NP_275327 |
| | | Methanococcus maripaludis S2 | MMP0880 | NP988000 |
| | | Methanococcus maripaludis C5 | MmarC5_0688 | YP001097214 |
| | | Methanococcus maripaludis C7 | MmarC7_0128 | YP_001329349 |
| | | Methanospaera stadtmanae DSM 3091 | Msp_0674 | YP_447715 |
| | | Methanopyrus kandleri AV19 | MK0782 | NP_614066 |
| | | Methanobrevibacter smithii ATCC35061 | Msm_0373 | YP001272946 |
| | | Methanococcus vannielii SB | Mevan_0040 | YP_001322567 |
| | | Methanococcus aeolicus Nankai 3 | Maeo_1484 | YP_001325672 |
| | | Methanosarcina acetivorans | MA3748* | NP_618621* |
| | | Methanospirillum hungatei JF-1 | Mhun_1797* | YP_503237* |
| | | Methanosphaera stadtmanae DSM 3091 | Msp_0674* | YP_447715* |
| | | Methanosaeta thermophila PT | Mthe_0855* | YP_843284* |
| | | Methanobrevibacter smithii ATCC 35061 | Msm_1298* | YP_001273871* |

References to gene and protein can be found via www.ncbi.nlm.nih.gov/((for listed gene/protein marked with an *: as available on 2 Mar. 2010, for the others: as available on 15 Apr. 2008).

In particular an enzyme may be used represented by any of the sequence ID's 4, 5, 6, 7, 8, 9, 10, 11, 12, 13, 261, 264, 267, 273, 276, 279, 282 (AksA), 14, 15, 16, 17, 18, 19, 20, 21, 22, 23, 186, 189, 192, 195, 225, 228, 231, 234 (AksD), 24, 25, 26, 27, 28, 29, 30, 31, 32, 33, 198, 201, 204, 207, 237, 240, 243, 246 (AksE), 34, 35, 36, 37, 38, 39, 40, 41, 42, 43, 210, 213, 216, 219, 222, 249, 252, 255, 258 (AksF), 44, 45, 46, 47, 48, 49, 50, 51, 52, 53 (AksA homologues), 54, 55, 56, 57, 58, 59, 60, 61 (AksD homologues), 62, 63, 64, 65, 66, 67 (AksF homologues), 69, 70, 71, 72, 73, 74, 75, 76, 77, 270 (AksA homologues).

AKP prepared in a method of the invention may further be used in the preparation of another compound, or be used as such, e.g. as a chemical for biochemical research or as a pH-buffer compound, e.g. for use in an preparative or analytical separation technique such as liquid chromatography or capillary electrophoresis. In particular, if desired, AKP may be used for the preparation of AAP, 5-FVA, 6-ACA or alpha-ketosuberic acid.

A method for preparing alpha-ketosuberic acid from AKP in a method of the invention comprises subjecting the AKP to C₁-elongation, using a biocatalyst as described herein. Thus, after AKP has been prepared by C₁-elongation, C1-elongation can be re-iterated once more, thereby forming alpha-ketosuberic acid from alpha-ketopimelic acid. To achieve this the same set of enzymes or homologues thereof as described above for the formation of AKP from AKA by C₁-elongation may be used. The formed alpha-ketosuberic acid can further be converted into 7-aminoheptanoic acid using the same concept as described herein for the conversion of AKP to 6-ACA, namely by using one or more biocatalysts selected from the group of decarboxylases, aminotransferases and amino acid dehydrogenases capable of catalysing a reaction step in a method of the invention. Alternatively, one or more of such subsequent reaction steps can be performed chemically. 7-Aminoheptanoic acid prepared in such way can then be cyclised to form the corresponding C₇-lactam (also referred to as azocan-2-one or zeta-aminoenantholactam) and/or polymerized directly or via the said C₇-lactam for the production of nylon-7 or copolymers thereof.

The inventors have realised that AKP can be converted into 6-ACA by a method wherein first AKP is decarboxylated to form 5-FVA after which 6-ACA can be prepared from 5-FVA using an amino transfer reaction or wherein first AKP is subjected to an amino transfer reaction to form AAP, after which 6-ACA can be prepared from AAP by a decarboxylation reaction.

In a preferred method for preparing 6-ACA, the preparation comprises a biocatalytic reaction in the presence of a biocatalyst capable of catalysing the decarboxylation of an alpha-keto acid or an amino acid (i.e. a compound comprising at least one carboxylic acid group and at least one amino group). An enzyme having such catalytic activity may therefore be referred to as an alpha-keto acid decarboxylase respectively an amino acid decarboxylase.

Said acid preferably is a diacid, wherein the said biocatalyst is selective towards the acid group next to the keto- or amino-group.

In general, a suitable decarboxylase has alpha-ketopimelate decarboxylase activity, capable of catalysing the conversion of AKP into 5-FVA or alpha-aminopimelate decarboxylase activity, capable of catalysing the conversion of AAP to 6-ACA.

An enzyme capable of decarboxylating an alpha-keto acid or an amino acid may in particular be selected from the group of decarboxylases (E.C. 4.1.1), preferably from the group of glutamate decarboxylases (EC 4.1.1.15), diaminopimelate decarboxylases (EC 4.1.1.20), aspartate 1-decarboxylases (EC 4.1.1.11), branched chain alpha-keto acid decarboxylases, alpha-ketoisovalerate decarboxylases (EC 1.2.4.4), alpha-ketoglutarate decarboxylases (EC 4.1.1.71), and pyruvate decarboxylases (EC 4.1.1.1).

One or more other suitable decarboxylases may in particular be selected amongst the group of oxalate decarboxylases (EC 4.1.1.2), oxaloacetate decarboxylases (EC 4.1.1.3), acetoacetate decarboxylases (EC 4.1.1.4), valine decarboxylases/leucine decarboxylases (EC 4.1.1.14), 3-hydroxyglutamate decarboxylases (EC 4.1.1.16), ornithine decarboxylases (EC 4.1.1.17), lysine decarboxylases (EC 4.1.1.18), arginine decarboxylases (EC 4.1.1.19), 2-oxoglutarate decarboxylases (EC 4.1.1.71), and diaminobutyrate decarboxylases (EC 4.1.1.86)

A decarboxylase may in particular be a decarboxylase of an organism selected from the group of squashes; cucumbers; yeasts; fungi, e.g. Saccharomyces cerevisiae, Candida flareri, Hansenula sp., Kluyveromyces marxianus, Rhizopus javanicus, Zymomonas mobilis, more in particular mutant 1472A from Zymomonas mobilis, and Neurospora crassa; mammals, in particular from mammalian brain; and bacteria. For instance glutamate decarboxylase or aspartate decarboxylase from Eschericia coli (E. coli) may be used, or glutamate decarboxylase from Neurospora crassa, Mycobacterium leprae, Clostridium perfringens, Lactobacillus brevis, Mycobacterium tuberculosis, Streptococcus or Lactococcus may be used. Examples of Lactococcus species from which the glutamate decarboxylase may originate in particular include Lactococcus lactis, such as Lactococcus lactis strain B1157, Lactococcus lactis IFPL730, more in particular Lactococcus lactis var. maltigenes (formerly named Streptococcus lactis var. maltigenes). An oxaloacetate decarboxylase from Pseudomonas may in particular be used.

In a preferred method of the invention, the preparation of 6-ACA comprises an enzymatic reaction in the presence of an enzyme capable of catalysing a transamination reaction in the presence of an amino donor, selected from the group of aminotransferases (E.C. 2.6.1).

In general, a suitable aminotransferase has 6-aminocaproic acid 6-aminotransferase activity, capable of catalysing the conversion of 5-FVA into 6-ACA oρ alpha-aminopimelate 2-aminotransferase activity, capable of catalysing the conversion of AKP into AAP.

The aminotransferase may in particular be selected amongst the group of beta-aminoisobutyrate: alpha-ketoglutarate aminotransferases, beta-alanine aminotransferases, aspartate aminotransferases, 4-amino-butyrate aminotransferases (EC 2.6.1.19), L-lysine 6-aminotransferase (EC 2.6.1.36), 2-aminoadipate aminotransferases (EC 2.6.1.39), 5-aminovalerate aminotransferases (EC 2.6.1.48), 2-aminohexanoate aminotransferases (EC 2.6.1.67), lysine: pyruvate 6-aminotransferases (EC 2.6.1.71) and aromatic amino acid aminotransferase (EC 2.6.1.57).

In an embodiment an aminotransferase may be selected amongst the group of alanine aminotransferases (EC 2.6.1.2), leucine aminotransferases (EC 2.6.1.6), alanine-oxo-acid aminotransferases (EC 2.6.1.12), beta-alanine-pyruvate aminotransferases (EC 2.6.1.18), (S)-3-amino-2-methylpropionate aminotransferases (EC 2.6.1.22), L,L-diaminopimelate aminotransferase (EC 2.6.1.83).

The aminotransferase may in particular be selected amongst aminotransferases from Vibrio, in particular Vibrio fluvialis; Pseudomonas, in particular Pseudomonas aeruginosa; Bacillus, in particular Bacillus weihenstephanensis; Mercurialis, in particular Mercurialis perennis, more in particular shoots of Mercurialis perennis; Asplenium, more in particular Asplenium unilaterale or Asplenium septentrionale; Ceratonia, more in particular Ceratonia siliqua; a mammal; or yeast, in particular Saccharomyces cerevisiae. In case the enzyme is of a mammal, it may in particular originate from mammalian kidney, from mammalian liver, from mammalian heart or from mammalian brain. For instance a suitable enzyme may be selected amongst the group of β-aminoisobutyrate: alpha-ketoglutarate aminotransferase from mammalian kidney, in particular beta-aminoisobutyrate: alpha-ketoglutarate aminotransferase from hog kidney; beta-alanine aminotransferase from mammalian liver, in particular beta-alanine aminotransferase from rabbit liver; aspartate aminotransferase from mammalian heart; in particular aspartate aminotransferase from pig heart; 4-amino-butyrate aminotransferase from mammalian liver, in particular 4-amino-butyrate aminotransferase from pig liver; 4-amino-butyrate aminotransferase from mammalian brain, in particular 4-aminobutyrate aminotransferase from human, pig, or rat brain; alpha-ketoadipate-glutamate aminotransferase from Neurospora, in particular alpha-ketoadipate: glutamate aminotransferase from Neurospora crassa; 4-amino-butyrate aminotransferase from E. coli, or alpha-aminoadipate aminotransferase from Thermus, in particular alpha-aminoadipate aminotransferase from Thermus thermophilus, and 5-aminovalerate aminotransferase from Clostridium in particular from Clostridium aminovalericum. A suitable 2-aminoadipate aminotransferase may e.g. be provided by Pyrobaculum islandicum.

In a specific embodiment, an aminotransferase is used comprising an amino acid sequence according to Sequence ID 2, 83, 86 or a homologue of any of these sequences.

In particular, the amino donor can be ammonia, ammonium ion, an amine or an amino acid. Suitable amines are primary amines and secondary amines. The amino acid may have a D- or L-configuration. Examples of amino donors are alanine, glutamate, isopropylamine, 2-aminobutane, 2-aminoheptane, phenylmethanamine, 1-phenyl-1-aminoethane, glutamine, tyrosine, phenylalanine, aspartate, beta-aminoisobutyrate, beta-alanine, 4-aminobutyrate, and alpha-aminoadipate.

In a further preferred embodiment, the method for preparing 6-ACA comprises a biocatalytic reaction in the presence of an enzyme capable of catalysing a reductive amination reaction in the presence of an ammonia source, selected from the group of oxidoreductases acting on the CH—NH₂ group of donors (EC 1.4), in particular from the group of amino acid dehydrogenases (E.C. 1.4.1). In general, a suitable amino acid dehydrogenase has 6-aminocaproic acid 6-dehydrogenase activity, catalysing the conversion of 5-FVA into 6-ACA or has alpha-aminopimelate 2-dehydrogenase activity, catalysing the conversion of AKP into AAP. In particular a suitable amino acid dehydrogenase be selected amongst the group of diaminopimelate dehydrogenases (EC 1.4.1.16), lysine 6-dehydrogenases (EC 1.4.1.18), glutamate dehydrogenases (EC 1.4.1.3; EC 1.4.1.4), and leucine dehydrogenases (EC 1.4.1.9).

In an embodiment, an amino acid dehydrogenase may be selected amongst an amino acid dehydrogenases classified as glutamate dehydrogenases acting with NAD or NADP as acceptor (EC 1.4.1.3), glutamate dehydrogenases acting with NADP as acceptor (EC 1.4.1.4), leucine dehydrogenases (EC 1.4.1.9), diaminopimelate dehydrogenases (EC 1.4.1.16), and lysine 6-dehydrogenases (EC 1.4.1.18).

An amino acid dehydrogenase may in particular originate from an organism selected from the group of Corynebacterium, in particular Corynebacterium glutamicum; Proteus, in particular Proteus vulgaris; Agrobacterium, in particular Agrobacterium tumefaciens; Geobacillus, in particular Geobacillus stearothermophilus; Acinetobacter, in particular Acinetobacter sp. ADP1; Ralstonia, in particular Ralstonia solanacearum; Salmonella, in particular Salmonella typhimurium; Saccharomyces, in particular Saccharomyces cerevisiae; Brevibacterium, in particular Brevibacterium flavum; and Bacillus, in particular Bacillus sphaericus, Bacillus cereus or Bacillus subtilis. For instance a suitable amino acid dehydrogenase may be selected amongst diaminopimelate dehydrogenases from Bacillus, in particular Bacillus sphaericus; diaminopimelate dehydrogenases from Brevibacterium sp.; diaminopimelate dehydrogenases from Corynebacterium, in particular diaminopimelate dehydrogenases from Corynebacterium glutamicum; diaminopimelate dehydrogenases from Proteus, in particular diaminopimelate dehydrogenase from Proteus vulgaris; lysine 6-dehydrogenases from Agrobacterium, in particular Agrobacterium tumefaciens, lysine 6-dehydrogenases from Geobacillus, in particular from Geobacillus stearothermophilus; glutamate dehydrogenases acting with NADH or NADPH as cofactor (EC 1.4.1.3) from Acinetobacter, in particular glutamate dehydrogenases from Acinetobactersp. ADP1; glutamate dehydrogenases (EC 1.4.1.3) from Ralstonia, in particular glutamate dehydrogenases from Ralstonia solanacearum; glutamate dehydrogenases acting with NADPH as cofactor (EC 1.4.1.4) from Salmonella, in particular glutamate dehydrogenases from Salmonella typhimurium; glutamate dehydrogenases (EC 1.4.1.4) from Saccharomyces, in particular glutamate dehydrogenases from Saccharomyces cerevisiae; glutamate dehydrogenases (EC 1.4.1.4) from Brevibacterium, in particular glutamate dehydrogenases from Brevibacterium flavum; and leucine dehydrogenases from Bacillus, in particular leucine dehydrogenases from Bacillus cereus or Bacillus subtilis.

In a specific embodiment, AKP is biocatalytically converted into 5-formylpentanoate (5-FVA) in the presence of a decarboxylase or other biocatalyst catalysing such conversion. A decarboxylase used in accordance with the invention may in particular be selected from the group of alpha-keto acid decarboxylases from Lactococcus lactis, Lactococcus lactis var. maltigenes or Lactococcus lactis subsp. cremoris; branched chain alpha-keto acid decarboxylases from Lactococcus lactis strain B1157 or Lactococcus lactis IFPL730; pyruvate decarboxylases from Saccharomyces cerevisiae, Candida flareri, Zymomonas mobilis, Hansenula sp., Rhizopus javanicus, Neurospora crassa, or Kluyveromyces marxianus; αλπηα-ketoglutarate decarboxylases from Mycobacterium tuberculosis; glutamate decarboxylases from E. coli, Lactobacillus brevis, Mycobacterium leprae, Neurospora crassa or Clostridium perfringens; and aspartate decarboxylases from E. coli.

Thereafter 5-FVA may be converted into 6-ACA. This can be done chemically: 6-ACA can be prepared in high yield by reductive amination of 5-FVA with ammonia over a hydrogenation catalyst, for example Ni on SiO₂/Al₂O₃ support, as described for 9-aminononanoic acid (9-aminopelargonic acid) and 12-aminododecanoic acid (12-aminolauric acid) in EP-A 628 535 or DE 4 322 065.

Alternatively, 6-ACA can be obtained by hydrogenation over PtO₂ of 6-oximocaproic acid, prepared by reaction of 5-FVA and hydroxylamine. (see e.g. F. O. Ayorinde, E. Y. Nana, P. D. Nicely, A. S. Woods, E. O. Price, C. P. Nwaonicha J. Am. Oil Chem. Soc. 1997, 74, 531-538 for synthesis of the homologous 12-aminododecanoic acid).

In an embodiment, the conversion of 5-FVA to 6-ACA may be performed biocatalytically in the presence of (i) an amino donor and (ii) an aminotransferase, an amino acid dehydrogenase or another biocatalyst capable of catalysing such conversion. In particular in such an embodiment the aminotransferase may be selected from the group of aminotransferases from Vibrio fluvialis, Pseudomonas aeruginosa or Bacillus weihenstephanensis; β-aminoisobutyrate:αλπηα-ketoglutarate aminotransferase from hog kidney; β-alanine aminotransferase from rabbit liver; aminotransferase from shoots from Mercurialis perennis; 4-aminobutyrate aminotransferase from pig liver or from human, rat, or pig brain; β-alanine aminotransferase from rabbit liver; and Llysine:alpha-ketoglutarate-ε-aminotransferase. In case an amino acid dehydrogenase is used, such amino acid dehydrogenase may in particular be selected from the group of lysine 6-dehydrogenases from Agrobacterium tumefaciens or Geobacillus stearothermophilus. Another suitable amino acid dehydrogenase may be selected from the group of diaminopimelate dehydrogenases from Bacillus sphaericus, Brevibacterium sp., Corynebacterium glutamicum, or Proteus vulgaris; from the group of glutamate dehydrogenases acting with NADH or NADPH as cofactor (EC 1.4.1.3) from Acinetobactersp. ADP1 or Ralstonia solanacearum; from the group of glutamate dehydrogenases acting with NADPH as cofactor (EC 1.4.1.4) from Salmonella typhimurium; from the group of glutamate dehydrogenases (EC 1.4.1.4) from Saccharomyces cerevisiae or Brevibacterium flavum; or from the group of leucine dehydrogenases from Bacillus cereus or Bacillus subtilis.

In a specific embodiment, AKP is chemically converted into 5-FVA. Efficient chemical decarboxylation of 2-keto carboxylic acid into the corresponding aldehyde can be performed by intermediate enamine formation using a secondary amine, for instance morpholine, under azeotropic water removal and simultaneous loss of CO₂, e.g. based on a method as described in Tetrahedron Lett. 1982, 23(4), 459-462. The intermediate terminal enamide is subsequently hydrolysed to the corresponding aldehyde. 5-FVA may thereafter be biocatalytically converted into 6-ACA by transamination in the presence of an aminotransferase or by enzymatic reductive amination by an amino acid dehydrogenase or another biocatalyst able of catalysing such conversion. Such aminotransferase or amino acid dehydrogenase may in particular be selected from the biocatalysts mentioned above when describing the conversion of 5-FVA to 6-ACA.

Alternatively, the conversion of 5-FVA to 6-ACA may be performed by a chemical method, e.g. as mentioned above.

In a specific embodiment, AKP is biocatalytically converted into AAP in the presence of (i) an aminotransferase, an amino acid dehydrogenase, or another biocatalyst capable of catalysing such conversion and (ii) an amino donor. Such aminotransferase used in accordance with the invention for the conversion of AKP to AAP may in particular be selected from the group of aspartate aminotransferases from pig heart; alpha-ketoadipate:glutamate aminotransferases from Neurospora crassa or yeast; aminotransferases from shoots from Mercurialis perennis; 4-aminobutyrate aminotransferases from E. coli; alpha-aminoadipate aminotransferases from Thermus thermophilus; aminotransferases from Asplenium septentrionale or Asplenium unilaterale; and aminotransferases from Ceratonia siliqua.

Suitable amino acid dehydrogenases may in particular be selected amongst the group of glutamate dehydrogenases acting with NADH or NADPH as cofactor (EC 1.4.1.3) from Acinetobactersp. ADP1 or Ralstonia solanacearum; glutamate dehydrogenases acting with NADPH as cofactor (EC 1.4.1.4) from Salmonella typhimurium, Saccharomyces cerevisiae, or Brevibacterium flavum; aminopimelate dehydrogenases from Bacillus sphaericus, Brevibacterium sp., Corynebacterium glutamicum, or Proteus vulgaris. Another suitable amino acid dehydrogenase may be selected from the group of lysine 6-dehydrogenases from Agrobacterium tumefaciens or Geobacillus stearothermophilus; or from the group of leucine dehydrogenases from Bacillus cereus or Bacillus subtilis.

Thereafter AAP may be chemically converted to 6-ACA by decarboxylation. This can be performed by heating in a high boiling solvent in the presence of a ketone or aldehyde catalyst. For example, amino acids are decarboxylated in good yields in cyclohexanol at 150-160° C. with 1-2 v/v % of cyclohexenone as described by M. Hashimoto, Y. Eda, Y. Osanai, T. Iwai and S. Aoki in Chem. Lett. 1986, 893-896. Similar methods are described in Eur. Pat. Appl. 1586553, 2005 by Daiso, and by S. D. Brandt, D. Mansell, S. Freeman, I. A. Fleet, J. F. Alder J. Pharm. Biomed. Anal. 2006, 41, 872-882.

Alternatively, the decarboxylation of AAP to 6-ACA may be performed biocatalytically in the presence of a decarboxylase or other biocatalyst catalysing such decarboxylation. The decarboxylase may be selected amongst decarboxylases capable of catalysing the decarboxylation of an alpha-amino acid. In particular, the decarboxylase may be selected from the group of glutamate decarboxylases from Curcurbita moschata, cucumber, yeast, or calf brain; and diaminopimelate decarboxylases (EC 4.1.1.20). A diaminopimelate decarboxylase may, e.g., be from an organism capable of synthesising lysine from diaminopimelate. Such organism may in particular be found amongst bacteria, archaea and plants. In particular, the diaminopimelate decarboxylase may be from a gram negative bacterium, for instance E. coli.

In a specific embodiment, AKP is chemically converted into AAP. AAP can be prepared from 2-oxopimelic acid by catalytic Leuckart-Wallach reaction as described for similar compounds. This reaction is performed with ammonium formate in methanol and [RhCp*cl₂]₂ as homogeneous catalyst (M. Kitamura, D. Lee, S. Hayashi, S. Tanaka, M. Yoshimura J. Org. Chem. 2002, 67, 8685-8687). Alternatively, the Leuckart-Wallach reaction can be performed with aqueous ammonium formate using [Ir^{III}Cp*(bpy)H₂O]SO₄ as catalyst as described by S. Ogo, K. Uehara and S. Fukuzumi in J. Am. Chem. Soc. 2004, 126, 3020-3021. Transformation of αλπηα-keto acids into (enantiomerically enriched) amino acids is also possible by reaction with (chiral) benzylamines and subsequent hydrogenation of the intermediate imine over Pd/C or Pd(OH)₂/C. See for example, R. G. Hiskey, R. C. Northrop J. Am. Chem. Soc. 1961, 83, 4798.

Thereafter AAP may be biocatalytically converted into 6-ACA, in the presence of a decarboxylase or another biocatalyst capable of performing such decarboxylation. Such decarboxylase may in particular be selected amongst the biocatalysts referred to above, when describing biocatalysts for the conversion of AAP to 6-ACA.

Alternatively, the conversion of AAP to 6-ACA may be performed by a chemical method, e.g. as mentioned above.

In a specific embodiment, AKP is biocatalytically converted into 5-FVA in the presence of a decarboxylase or other biocatalyst capable of catalysing such conversion and 5-FVA is thereafter converted into 6-ACA in the presence of an aminotransferase, amino acid dehydrogenase, or other biocatalyst capable of catalysing such conversion. Decarboxylases suitable for these reactions may in particular be selected from the group of decarboxylases mentioned above, when describing the biocatalytic conversion of AKP into 5-FVA. A suitable aminotransferase or amino acid dehydrogenase for the conversion of 5-FVA may in particular be selected from those mentioned above, when describing the biocatalytic conversion of 5-FVA to 6-ACA.

In a specific embodiment, AKP is biocatalytically converted into AAP in the presence of an aminotransferase, amino acid dehydrogenase, or other biocatalyst capable of catalysing such conversion and AAP is thereafter converted into 6-ACA in the presence of a decarboxylase. Enzymes suitable for these reactions may in particular be selected from the group of aminotransferases, amino acid dehydrogenases, and decarboxylases which have been described above when describing the biocatalytic conversion of AKP into AAP and the biocatalytic conversion of AAP into 6-ACA respectively.

In another embodiment of the invention, 6-ACA—prepared from AKP made in a method according to the invention—is converted into diaminohexane. This may be accomplished by reducing the acid group to form an aldehyde group, and transaminating the thus formed aldehyde group, thereby providing an aminogroup, yielding diaminohexane. This may be accomplished chemically or biocatalytically. In a preferred method of the invention, the preparation comprises a biocatalytic reaction in the presence of a biocatalyst capable of catalysing the reduction of the acid to form an aldehyde group and/or a biocatalytic reaction in the presence of a biocatalyst capable of catalysing said transamination, in the presence of an amino donor, e.g. an amino donor as described elsewhere herein.

A biocatalyst capable of catalysing the reduction of the acid group to form an aldehyde group may in particular comprise an enzyme selected from the group of oxidoreductases (EC 1.2.1), preferably from the group of aldehyde dehydrogenases (EC 1.2.1.3, EC 1.2.1.4 and EC 1.2.1.5), acetaldehyde dehydrogenase (acetylating) (EC 1,2,1,10); aspartate-semialdehyde dehydrogenase (EC 1.2.1.11); malonate-semialdehyde dehydrogenase (EC 1.2.1.15); and succinate-semialdehyde dehydrogenase (EC 1.2.1.16 and EC 1.2.1.24).

The oxidoreductase may in principle be obtained or derived from any organism. The organism may be prokaryotic or eukaryotic. In particular the organism can be selected from bacteria, archaea, yeasts, fungi, protists, plants and animals (including human).

In an embodiment the oxidoreductase, in particular the aldehyde dehydrogenase, is obtained or derived from a bacterium selected from the group of Acinetobacter (in particular Acinetobacter baumanii and Acinetobacter sp. NCIMB9871), Azospirillum (in particular Azospirillum brasilense) Ralstonia, Bordetella, Burkholderia, Methylobacterium, Xanthobacter, Sinorhizobium, Rhizobium, Nitrobacter, Brucella (in particular B. melitensis), Pseudomonas, Agrobacterium (in particular Agrobacterium tumefaciens), Bacillus, Listeria, Alcaligenes, Corynebacterium, Escherichia, and Flavobacterium.

In an embodiment, the oxidoreductase, in particular the aldehyde dehydrogenase, is obtained or derived from an organism selected from the group of yeasts and fungi, in particular from the group of Aspergillus (in particular A. niger and A. nidulans) and Penicillium (in particular P. chrysogenum).

In an embodiment, the oxidoreductase, in particular the aldehyde dehydrogenase, is obtained or derived from a plant, in particular Arabidopsis, more in particular A. thaliana.

A biocatalyst capable of catalysing the transamination reaction in the conversion to diaminohexane may in particular comprise an enzyme selected from the group of aminotransferases (E.C. 2.6.1), e.g. found in an organism as described elsewhere herein.

Reaction conditions in a method of the invention may be chosen depending upon known conditions for the biocatalyst, in particular the enzyme, the information disclosed herein and optionally some routine experimentation.

In principle, the pH of the reaction medium used may be chosen within wide limits, as long as the biocatalyst is active under the pH conditions. Alkaline, neutral or acidic conditions may be used, depending on the biocatalyst and other factors. In case the method includes the use of a micro-organism, e.g. for expressing an enzyme catalysing a method of the invention, the pH is selected such that the micro-organism is capable of performing its intended function or functions. The pH may in particular be chosen within the range of four pH units below neutral pH and two pH units above neutral pH, i.e. between pH 3 and pH 9 in case of an essentially aqueous system at 25° C. A system is considered aqueous if water is the only solvent or the predominant solvent (>50 wt. %, in particular >90 wt. %, based on total liquids), wherein e.g. a minor amount (<50 wt. %, in particular <10 wt. %, based on total liquids) of alcohol or another solvent may be dissolved (e.g. as a carbon source) in such a concentration that micro-organisms which may be present remain active. In particular in case a yeast and/or a fungus is used, acidic conditions may be preferred, in particular the pH may be in the range of pH 3 to pH 8, based on an essentially aqueous system at 25° C. If desired, the pH may be adjusted using an acid and/or a base or buffered with a suitable combination of an acid and a base.

In principle, the incubation conditions can be chosen within wide limits as long as the biocatalyst shows sufficient activity and/or growth. This includes aerobic, micro-aerobic, oxygen limited and anaerobic conditions.

Anaerobic conditions are herein defined as conditions without any oxygen or in which substantially no oxygen is consumed by the biocatalyst, in particular a micro-organism, and usually corresponds to an oxygen consumption of less than 5 mmol/l.h, in particular to an oxygen consumption of less than 2.5 mmol/l.h, or less than 1 mmol/l.h.

Aerobic conditions are conditions in which a sufficient level of oxygen for unrestricted growth is dissolved in the medium, able to support a rate of oxygen consumption of at least 10 mmol/l.h, more preferably more than 20 mmol/l.h, even more preferably more than 50 mmol/l.h, and most preferably more than 100 mmol/l.h.

Oxygen-limited conditions are defined as conditions in which the oxygen consumption is limited by the oxygen transfer from the gas to the liquid. The lower limit for oxygen-limited conditions is determined by the upper limit for anaerobic conditions, i.e. usually at least 1 mmol/l.h, and in particular at least 2.5 mmol/l.h, or at least 5 mmol/l.h. The upper limit for oxygen-limited conditions is determined by the lower limit for aerobic conditions, i.e. less than 100 mmol/l.h, less than 50 mmol/l.h, less than 20 mmol/l.h, or less than to 10 mmol/l.h.

Whether conditions are aerobic, anaerobic or oxygen limited is dependent on the conditions under which the method is carried out, in particular by the amount and composition of ingoing gas flow, the actual mixing/mass transfer properties of the equipment used, the type of micro-organism used and the micro-organism density.

In a preferred method of the invention, at least the preparation of AKP is carried out under fermentative conditions.

In principle, the temperature used is not critical, as long as the biocatalyst, in particular the enzyme, shows substantial activity. Generally, the temperature may be at least 0° C., in particular at least 15° C., more in particular at least 20° C. A desired maximum temperature depends upon the biocatalyst. In general such maximum temperature is known in the art, e.g. indicated in a product data sheet in case of a commercially available biocatalyst, or can be determined routinely based on common general knowledge and the information disclosed herein. The temperature is usually 90° C. or less, preferably 70° C. or less, in particular 50° C. or less, more in particular or 40° C. or less.

In particular if a biocatalytic reaction is performed outside a host organism, a reaction medium comprising an organic solvent may be used in a high concentration (e.g. more than 50%, or more than 90 wt. %), in case an enzyme is used that retains sufficient activity in such a medium.

A compound prepared in a method of the invention can be recovered from the medium in which it has been prepared. Recovery conditions may be chosen depending upon known conditions for recovery the specific compound, the information disclosed herein and optionally some routine experimentation.

A heterologous cell comprising one or more enzymes for catalysing a reaction step in a method of the invention can be constructed using molecular biological techniques, which are known in the art per se. For instance, such techniques can be used to provide a vector which comprises one or more genes encoding one or more of said biocatalysts. A vector comprising one or more of such genes can comprise one or more regulatory elements, e.g. one or more promoters, which may be operably linked to a gene encoding an biocatalyst.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polym erase, transcription initiation sites and any other DNA sequences, including, but not limited to transcription factor binding sites, repressor and activator protein binding sites, and any other sequences of nucleotides known to one of skilled in the art to act directly or indirectly to regulate the amount of transcription from the promoter. A “constitutive” promoter is a promoter that is active under most environmental and developmental conditions. An “inducible” promoter is a promoter that is active under environmental or developmental regulation. The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

The promoter that could be used to achieve the expression of the nucleotide sequences coding for an enzyme for use in a method of the invention, in particular an aminotransferase, an amino acid dehydrogenase or a decarboxylase, such as described herein above may be native to the nucleotide sequence coding for the enzyme to be expressed, or may be heterologous to the nucleotide sequence (coding sequence) to which it is operably linked. Preferably, the promoter is homologous, i.e. endogenous to the host cell.

If a heterologous promoter (to the nucleotide sequence encoding for the enzyme of interest) is used, the heterologous promoter is preferably capable of producing a higher steady state level of the transcript comprising the coding sequence (or is capable of producing more transcript molecules, i.e. mRNA molecules, per unit of time) than is the promoter that is native to the coding sequence. Suitable promoters in this context include both constitutive and inducible natural promoters as well as engineered promoters, which are well known to the person skilled in the art.

A “strong constitutive promoter” is one which causes mRNAs to be initiated at high frequency compared to a native host cell. Examples of such strong constitutive promoters in Gram-positive micro-organisms include SP01-26, SP01-15, veg, pyc (pyruvate carboxylase promoter), and amyE.

Examples of inducible promoters in Gram-positive micro-organisms include, the IPTG inducible Pspac promoter, the xylose inducible PxyIA promoter.

Examples of constitutive and inducible promoters in Gram-negative microorganisms include, but are not limited to, tac, tet, trp-tet, lpp, lac,lpp-lac, lacIq, T7, T5, T3, gal, trc, ara (P_{BAD}), SP6, λ-P_{R} and λ-P_{L}.

Promoters for (filamentous) fungal cells are known in the art and can be, for example, the glucose-6-phosphate dehydrogenase gpdA promoters, protease promoters such as pepA, pepB, pepC, the glucoamylase glaA promoters, amylase amyA, amyB promoters, the catalase catR or catA promoters, glucose oxidase goxC promoter, beta-galactosidase lacA promoter, alpha-glucosidase aglA promoter, translation elongation factor tefA promoter, xylanase promoters such as xlnA, xlnB, xlnC, xlnD, cellulase promoters such as eglA, egIB, cbhA, promoters of transcriptional regulators such as areA, creA, xlnR, pacC, prtT, etc or any other, and can be found among others at the NCBI website (http://www.ncbi.nlm.nih.gov/entrez/

The invention also relates to a novel heterologous cell which may provide one or more biocatalysts capable of catalysing at least one reaction step in the preparation of AKP, and optionally in the preparation of a further compound from AKP, such as 5-FVA, AAP, 6-ACA, diaminohexane or caprolactam. The invention also relates to a novel vector comprising one or more genes encoding for one or more enzymes capable of catalysing at least one reaction step in the preparation of AKP, and optionally in the preparation of a further compound from AKP, such as 5-FVA, AAP, 6-ACA, diaminohexane or caprolactam. One or more suitable genes may in particular be selected amongst genes encoding an enzyme as mentioned herein above. In particular, at least one of such genes is heterologous to the host organism.

In a particularly advantageous embodiment the heterologous cell or the vector comprises an AksD, an AksE, an AksF and an NifV gene. In a further particularly advantageous embodiment the heterologous cell additionally comprises an AksA gene. Preferred AksA, AksD, AksE and AksF genes are from M. jannashii, from S. cerevisiae, from M. Maripaludis, from Methanosarcina acetivorans, from Methanospirillum hungatei or from E. coli.

The NifV gene is preferably from Azotobacter vinelandii. In a particularly preferred embodiment, the NifV gene comprises a sequence represented by SEQ ID NO: 149, or a functional analogue thereof.

Regarding the genes selected from the group of AksA, AksD, AksE and AksF genes, preferably, the genome of a cell (used) according to the invention comprises at least one nucleic acid sequence according to any of the sequences selected from the group of SEQ ID NO's 145, 146, 147, 148; SEQ ID NO's 167, 168, 169, 170, 171, 172, 173, 174; SEQ ID NO's 177, 178, 179, 180, 181, 182, 183, 184; SEQ ID NO's 224, 226, 236, 238, 248, 250, 260, 262; SEQ ID NO's 227, 229, 239, 241, 251, 253, 263, 265; SEQ ID NO's; 194, 196, 206, 208, 221, 223, 281, 283; SEQ ID NO's; 188, 190, 200, 202, 215, 217, 272, 274 and functional analogues thereof. In a specific embodiment, the cell comprises an an AksA, an AksD, an AksE and an AksF gene selected from the group of sequences. In a further specific embodiment, the cell comprises an NifV gene comprising a sequence represented by SEQ ID NO: 149 or a functional analogue thereof, an AksD, an AksE and an AksF gene selected from the group of sequences.

In a particularly preferred embodiment, one, two three or each of these genes selected from the group of AksA, AksD, AksE and AksF genes comprise a sequence selected from the sequences represented by SEQ ID NO: 145, 146, 147, 148 respectively (AksA, D, E and F respectively) and functional analogous thereof. In a further particularly preferred embodiment, one, two three or each of these genes comprise a sequence represented by respectively SEQ ID NO: 167, 168, 169, 170 respectively (AksA, D, E and F respectively) and functional analogous thereof. In a particularly preferred embodiment, one, two three or each of these genes selected from the group of AksA, AksD, AksE and AksF genes comprise a sequence selected from the sequences represented by represented by SEQ ID NO: 260, 224, 236, 248, respectively (AksA, D, E and F respectively) and functional analogous thereof.

In a particularly preferred embodiment, one, two three or each of these genes selected from the group of AksA, AksD, AksE and AksF genes comprise a sequence selected from the sequences represented by represented by SEQ ID NO: 262, 226, 238, 250, respectively (AksA, D, E and F respectively) and functional analogous thereof.

In a particularly preferred embodiment, one, two three or each of these genes selected from the group of AksA, AksD, AksE and AksF genes comprise a sequence selected from the sequences represented by represented by SEQ ID NO: 263, 227, 239, 251, respectively (AksA, D, E and F respectively) and functional analogous thereof.

In a particularly preferred embodiment, one, two three or each of these genes comprise a sequence selected from the sequences represented by represented by SEQ ID NO: 265, 229, 241, 253, respectively (AksA, D, E and F respectively) and functional analogous thereof.

In a particularly preferred embodiment, one, two, three or each of these genes selected from the group of AksA, AksD, AksE and AksF genes comprise a sequence selected from the sequences represented by represented by SEQ ID NO: 281, 194, 206, 221 respectively (AksA, D, E and F respectively) and functional analogous thereof.

In a particularly preferred embodiment, one, two three or each of these genes selected from the group of AksA, AksD, AksE and AksF genes comprise a sequence selected from the sequences represented by represented by SEQ ID NO: 283, 196, 208, 223, respectively (AksA, D, E and F respectively) and functional analogous thereof.

In a particularly preferred embodiment, one, two three or each of these genes selected from the group of AksA, AksD, AksE and AksF genes comprise a sequence selected from the sequences represented by represented by SEQ ID NO: 272, 188, 200, 215 respectively (AksA, D, E and F respectively) and functional analogous thereof.

In a particularly preferred embodiment, one, two three or each of these genes selected from the group of AksA, AksD, AksE and AksF genes comprise a sequence selected from the sequences represented by represented by SEQ ID NO: 274, 190, 202, 217 respectively (AksA, D, E and F respectively) and functional analogous thereof.

In yet a further particularly preferred embodiment, one, two three or each of these genes selected from the group of AksA, AksD, AksE and AksF genes comprise a sequence selected from the sequences represented by respectively SEQ ID NO: 161, 162, 163, 164, 165, 166, 167, 168, 169, 170, 171, 172, 173, 174 respectively (AksA, D, E and F respectively) and functional analogous thereof. In yet a further particularly preferred embodiment, one, two three or each of these genes comprise a sequence selected from the sequences represented by respectively SEQ ID NO: 177, 178, 179, 180 respectively (AksA, D, E and F respectively) and functional analogous thereof.

In yet a further particularly preferred embodiment, one, two three or each of these genes selected from the group of AksA, AksD, AksE and AksF genes comprise a sequence selected from the sequences represented by respectively SEQ ID NO: 260, 224, 236, 248, respectively (AksA, D, E and F respectively) and functional analogous thereof.

In yet a further particularly preferred embodiment, one, two three or each of these genes selected from the group of AksA, AksD, AksE and AksF genes comprise a sequence selected from the sequences represented by respectively SEQ ID NO: 263, 227, 239, 251, respectively (AksA, D, E and F respectively) and functional analogous thereof.

In yet a further particularly preferred embodiment, one, two three or each of these genes selected from the group of AksA, AksD, AksE and AksF genes comprise a sequence selected from the sequences represented by respectively SEQ ID NO: 281, 194, 206, 221, respectively (AksA, D, E and F respectively) and functional analogous thereof.

In yet a further particularly preferred embodiment, one, two three or each of these genes comprise a sequence selected from the sequences represented by respectively SEQ ID NO: 272, 188, 200, 215, respectively (AksA, D, E and F respectively) and functional analogous thereof.

In a particularly preferred embodiment, the genome of the cell comprises a nucleic acid sequence represented by sequence ID145, or a functional analogue thereof, a nucleic acid sequence represented by sequence ID146, or a functional analogue thereof, a nucleic acid sequence represented by sequence ID147, or a functional analogue thereof, a nucleic acid sequence represented by sequence ID148, or a functional analogue thereof, and a nucleic acid sequence represented by sequence ID149, or a functional analogue thereof.

In a particularly preferred embodiment, the genome of the cell comprises a nucleic acid sequence represented by sequence ID146, or a functional analogue thereof, a nucleic acid sequence represented by sequence ID147, or a functional analogue thereof, a nucleic acid sequence represented by sequence ID148, or a functional analogue thereof, and a nucleic acid sequence represented by sequence ID149, or a functional analogue thereof.

In a particularly preferred embodiment, the genome of the cell comprises a nucleic acid sequence represented by sequence ID172, or a functional analogue thereof, a nucleic acid sequence represented by sequence ID173, or a functional analogue thereof, a nucleic acid sequence represented by sequence I D174, or a functional analogue thereof, and a nucleic acid sequence represented by sequence ID149, or a functional analogue thereof.

In a particularly preferred embodiment, the genome of the cell comprises a nucleic acid sequence represented by sequence ID 224, or a functional analogue thereof, a nucleic acid sequence represented by sequence ID 236, or a functional analogue thereof, a nucleic acid sequence represented by sequence ID 248, or a functional analogue thereof, and a nucleic acid sequence represented by sequence ID 149, or a functional analogue thereof.

In a particularly preferred embodiment, the genome of the cell comprises a nucleic acid sequence represented by sequence ID 227, or a functional analogue thereof, a nucleic acid sequence represented by sequence ID 239, or a functional analogue thereof, a nucleic acid sequence represented by sequence ID 251, or a functional analogue thereof, and a nucleic acid sequence represented by sequence ID 149, or a functional analogue thereof.

In a particularly preferred embodiment, the genome of the cell comprises a nucleic acid sequence represented by sequence ID194, or a functional analogue thereof, a nucleic acid sequence represented by sequence ID206, or a functional analogue thereof, a nucleic acid sequence represented by sequence ID221, or a functional analogue thereof, and a nucleic acid sequence represented by sequence ID 149, or a functional analogue thereof.

In a particularly preferred embodiment, the genome of the cell comprises a nucleic acid sequence represented by sequence ID188, or a functional analogue thereof, a nucleic acid sequence represented by sequence ID200, or a functional analogue thereof, a nucleic acid sequence represented by sequence ID215, or a functional analogue thereof, and a nucleic acid sequence represented by sequence ID 149, or a functional analogue thereof.

In a particularly preferred embodiment, the genome of the cell comprises a nucleic acid sequence represented by sequence ID177, or a functional analogue thereof, a nucleic acid sequence represented by sequence ID178, or a functional analogue thereof, a nucleic acid sequence represented by sequence ID179, or a functional analogue thereof, a nucleic acid sequence represented by sequence ID180, or a functional analogue thereof, and a nucleic acid sequence represented by sequence ID149, or a functional analogue thereof.

In a particularly preferred embodiment, the genome of the cell comprises a nucleic acid sequence represented by sequence ID224, or a functional analogue thereof, a nucleic acid sequence represented by sequence ID236, or a functional analogue thereof, a nucleic acid sequence represented by sequence ID248, or a functional analogue thereof, a nucleic acid sequence represented by sequence ID260, or a functional analogue thereof, and a nucleic acid sequence represented by sequence ID149, or a functional analogue thereof.

In a particularly preferred embodiment, the genome of the cell comprises a nucleic acid sequence represented by sequence ID227, or a functional analogue thereof, a nucleic acid sequence represented by sequence ID239, or a functional analogue thereof, a nucleic acid sequence represented by sequence ID251, or a functional analogue thereof, a nucleic acid sequence represented by sequence ID263, or a functional analogue thereof, and a nucleic acid sequence represented by sequence ID149, or a functional analogue thereof.

In a particularly preferred embodiment, the genome of the cell comprises a nucleic acid sequence represented by sequence ID194, or a functional analogue thereof, a nucleic acid sequence represented by sequence ID206, or a functional analogue thereof, a nucleic acid sequence represented by sequence ID221, or a functional analogue thereof, a nucleic acid sequence represented by sequence ID281, or a functional analogue thereof, and a nucleic acid sequence represented by sequence ID149, or a functional analogue thereof.

In a particularly preferred embodiment, the genome of the cell comprises a nucleic acid sequence represented by sequence ID188, or a functional analogue thereof, a nucleic acid sequence represented by sequence ID200, or a functional analogue thereof, a nucleic acid sequence represented by sequence ID215, or a functional analogue thereof, a nucleic acid sequence represented by sequence ID272, or a functional analogue thereof, and a nucleic acid sequence represented by sequence ID149, or a functional analogue thereof.

Good results have been achieved with a E. coli host cell of which the genome comprises heterologous nucleic acid sequences, represented by SEQ ID No's: 149, 167, 168, 169 and 170.

Good results have been achieved with a E. coli host cell of which the genome comprises heterologous nucleic acid sequences, represented by SEQ ID No's: 149, 168, 169 and 170.

Good results have been achieved with a S. cerevisiae host cell of which the genome comprises heterologous nucleic acid sequences, represented by sequence ID's 149, 172, 173 and 174.

Good results have been achieved with a E. coli host cell of which the genome comprises heterologous nucleic acid sequences, represented by SEQ ID No's: 149, 177, 178, 179, 180.

Good results have been achieved with a E. coli host cell of which the genome comprises heterologous nucleic acid sequences, represented by SEQ ID No's: 149, 224, 236, 248.

Good results have been achieved with a E. coli host cell of which the genome comprises heterologous nucleic acid sequences, represented by SEQ ID No's: 149, 227, 239, 251.

Good results have been achieved with a E. coli host cell of which the genome comprises heterologous nucleic acid sequences, represented by SEQ ID No's: 149, 194, 206, 221.

Good results have been achieved with a E. coli host cell of which the genome comprises heterologous nucleic acid sequences, represented by SEQ ID No's: 149, 188, 200, 251.

The heterologous cell may in particular be a cell as mentioned above when describing the biocatalyst.

In particular, a heterologous cell according to the invention, comprises one or more heterologous nucleic acid sequences (which may be part of one or more vectors) encoding one or more heterologous enzymes capable of catalysing at least one reaction step in the preparation of α-ketopimelic acid from α-ketoglutaric acid or in the conversion of AKP to AAP, 6-ACA, 5-FVA, caprolactam, or diaminohexane.

In a specific embodiment, the cell comprises one or more nucleic acid sequences, which may be homologous or heterologous, encoding an enzyme system capable of catalysing the conversion of alpha-ketoglutaric acid into alpha-ketoadipic acid, wherein said enzyme system forms part of the AAA biosynthetic pathway for lysine biosynthesis, such as described in more detail above.

The heterologous cell is preferably free of aminotransferase activity capable of catalysing the conversion of -alpha-ketoadipate into alpha-aminoadipate. If naturally present in the cell, the activity may be removed, decreased or modified by inactivation, modification or deletion of the gene or genes encoding such enzymes in the cells DNA. This activity may originate from one or more biocatalysts. These may also be modified e.g. by molecular evolution or rational design to not possess any undesired activity any more but to retain any desired activity (e.g. any activity in the context of the invention or an activity required for metabolism of the host cell).

The heterologous cell is preferably free of any enzyme(s) which can degrade or convert AKP, 5-FVA, AAP, 6-ACA, caprolactam or diaminohexane into any undesired side product. If any such activity e.g. as part of a caprolactam degradation pathway is identified this activity can be removed, decreased or modified as described herein above.

Preferably, the cell comprises one or more heterologous nucleic acid sequences encoding one or more enzymes catalysing the C₁-elongation of alpha-ketoglutaric acid into alpha-ketoadipic acid and/or C₁-elongation of alpha-ketoadipic acid into alpha-ketopimelic acid. Suitable nucleic acid sequences may in particular be selected amongst nucleic acid sequences encoding an Aks enzyme or an homologue thereof, such as identified above.

In particular in case the cell is intended to be used for preparing AKP, which in turn is to be converted into a further product, such as 5-FVA or AAP, which in turn may be further converted to 6-ACA, caprolactam or diaminohexane, it is preferred that the heterologous cell comprises a nucleic acid sequence encoding an enzyme catalysing such conversion. This may be advantageous, for instance in that at least some enzymes catalysing C₁-elongation, which may be active in the cell may be capable of catalysing the undesired elongation of AKP. By expressing an enzyme capable of catalysing the conversion of AKP into a desired product, such as 5-FVA or AAP, such as a decarboxylase or an aminotransferase, in the cell, it is contemplated that such undesired elongation may be reduced or substantially avoided, also if the enzyme or enzymes catalysing the elongation are in principle capable of using AKP as a substrate.

It is noted that some of the enzymes involved in C₁-elongations e.g. in M. jannashii or A. vinelandii have relaxed substrate specificity and are able to convert substrates of different carbon length. It is known for many enzymes that they have a relaxed substrate specificity which allows them to convert unnatural substrates. In order to improve the efficiency of a heterologous cell (used in a method) according to the invention, it is particularly preferred to provide an enzyme system capable of catalysing a reaction step in the preparation of AKP from AKG that shows a high catalytic activity towards the elongation of AKG into AKA and/or the elongation of AKA into AKP, yet a low catalytic activity towards the further elongation of AKP. (A nucleic acid sequence coding for) one or more enzymes capable of catalysing a reaction step in the preparation of AKP from AKG may be modified by a technique such as described above in order to increase the reaction specificity with respect to elongation of AKG and/or AKA, and/or (a nucleic acid sequence coding for) such enzyme may be modified such that the binding affinity for AKP (as a substrate) is reduced such that the catalytic activity with respect to the elongation of AKP is reduced.

Such modification may involve molecular evolution to create diversity followed by screening for desired mutants and/or rational engineering of substrate binding pockets. Techniques to modify the substrate specificity of an enzyme used in a method of the invention may be based on those described in the art. In particular, an AksA enzyme or homologue thereof, capable of catalysing “reaction a” of the C₁-elongation may be evolved such that the catalytic activity with respect to catalysing the elongation of AKP to alpha-ketosuberate is reduced, relatively to the catalytic activity with respect to catalysing the elongation of AKA to AKP and/or AKG to AKA. Preferably, such enzyme shows no substantial catalytic activity with respect to catalysing the elongation of AKP to alpha-ketosuberate. It is thought that in particular the enzyme catalysing “reaction a” controls the maximum chain length obtainable by the C₁-elongation, unless of course the AKP is intended to serve as a substrate in the preparation of alpha-ketosuberate.

For instance, rational engineering employing structural and sequence information to design specific mutations has been utilised to modify the substrate specificity of the acyl transferase domain 4 from the erythromycin polyketide synthase to accept alternative acyl donors. It has been shown that modifying the proposed substrate binding site resulted in a modified binding pocket able to accommodate alternative substrates resulting in a different product ratio (Reeves, C. D.; Murli, S.; Ashley, G. W.; Piagentini, M.; Hutchinson, C. R.; McDaniel, R. Biochemistry 2001, 40(51), 15464-15470). Both rational design and molecular evolution approaches have been used to alter the substrate specificity of the biocatalyst BM3 resulting in a large number of mutants capable of oxidizing a large variety of different alkenes, cycloalkenes, arenes and heteroarenes instead or in addition to the natural substrate of medium chain fatty acids (e.g. myristic acid) (Peters, M. W.; Meinhold, P.; Glieder, A.; Arnold, F. H. Journal of the American Chemical Society 2003, 125(44), 13442-13450; Appel, D.; Lutz-Wahl, S.; Fischer, P.; Schwaneberg, U.; Schmid, R. D. Journal of Biotechnology 2001, 88(2), 167-171 and references therein).

In an embodiment, the heterologous cell comprises a heterologous nucleic acid sequence encoding a homocitrate synthase that has been evolved from a homocitrate synthase, which accepted alpha-ketoglutarate as a substrate but for which alpha-ketoadipate was not a suitable substrate, to also accept alpha-ketoadipate as a substrate. Such enzyme may in particular be a fungal enzyme or bacterial enzyme involved in lysine biosynthesis via the AAA pathway e.g. from Penicillium, Cephalosporium, Ustilago, Cephalosporium, Paelicomyces, Trichophytum, Phanerochaete, Emericella, Aspergillus, Yarrowoa, Schizosaccharomyces, Pichia, Hansenula, Klyuveromyces, Candida, Saccharomyces, Thermus, or Deinococcus, or from nitrogen fixing bacteria, e.g. Azotobacter, Frankia, Synecchocystis, Anabaena, Microcyctis, Rhizobium, Bradyrhizobium, Klebsiella, or Pseudomonas. In particular an enzyme such as NifV from Azotobacter vinelandii may be used, which was demonstrated to have initial activity on AKA (Zheng, L.; White, R. H.; Dean, D. R. The Journal of Bacteriology 1997, 179(18), 5963-5966). In Sequence ID 149 a gene encoding said enzyme is shown.

The heterologous cell may in particular comprise a nucleic acid sequence encoding an Aks enzyme or homologue thereof, such as identified above, more in particular the cell may at least comprise a nucleic acid sequence encoding an Aks enzyme or a homologue thereof, preferably a nucleic acid sequence encoding an enzyme may be used represented by any of the sequence ID's 4, 5, 6, 7, 8, 9, 10, 11, 12, 13 44, 45, 46, 47, 48, 49, 50, 51, 52, 53, 69, 70, 71, 72, 73, 74, 75, 76, 77, 261, 264, 267, 270, 273, 276, 279, 282 or a homologue thereof.

In a further preferred embodiment the cell comprises at least one nucleic acid sequence encoding an enzyme represented by any of the sequence ID's 14, 15, 16, 17, 18, 19, 20, 21, 22, 23, 54, 55, 56, 57, 58, 59, 60, 61, 186, 189, 192, 195, 225, 228, 231, 234 or a homologue thereof.

In a further preferred embodiment the cell comprises at least one nucleic acid sequence encoding an enzyme represented by any of the sequence ID's 24, 24, 25, 26, 27, 28, 29, 30, 31, 32, 33, 198, 201, 204, 207, 237, 240, 243, 246 or a homologue thereof.

In a further preferred embodiment the cell comprises at least one nucleic acid sequence encoding an enzyme represented by any of the sequence ID's 34, 35, 36, 37, 38, 39, 40, 41, 42, 43, 62, 63, 64, 65, 66, 67, 210, 213, 216, 219, 222, 249, 252, 255, 258 or a homologue thereof.

In an embodiment, the heterologous organism is based on a host cell that has the AAA pathway for lysine biosynthesis, wherein a homocitrate synthase, capable of catalysing “reaction a” in the C₁-elongation (such as AksA or a homologue thereof) may be heterologously expressed. Such homocitrate synthase preferably is capable of selectively catalysing a reaction step in the elongation of AKG and/or AKA (reaction a), without substantially catalysing the elongation of AKP. In such a case it may be beneficial to delete any endogenous homo citrate synthase, in particular if it is capable of catalysing “reaction a” in the elongation reaction of AKP. Such a host cell may then effectively contain one or more homo citrate synthases functionally active in the C₁-elongation of AKG to AKA and/or AKA to AKP. Further reactions to realise the elongation of AKG and/or AKA may then be catalysed by endogenous enzymes, such as those enzymes involved in the aminoadipate pathway.

In an embodiment, the heterologous cell comprises (a recombinant vector comprising) a nucleic acid sequence encoding an enzyme with alpha-ketopimelic acid aminotransferase activity and/or a nucleic acid sequence encoding an enzyme with alpha-aminopimelic acid decarboxylase activity.

In a preferred embodiment, a heterologous cell according to the invention comprises a nucleic acid sequence encoding an enzyme with AKP decarboxylase activity and/or a nucleic acid sequence encoding an enzyme with 5-FVA aminotransferase activity. In a preferred embodiment, a heterologous cell according to the invention comprises a nucleic acid sequence encoding an enzyme with alpha-aminopimelate 2-dehydrogenase or AKP aminotransferase activity and/or a nucleic acid sequence encoding an enzyme with alpha-aminopimelate decarboxylase activity.

In a preferred embodiment, a heterologous cell according to the invention comprises a nucleic acid sequence encoding an enzyme with 6-aminocaproic acid 6-dehydrogenase activity and optionally a nucleic acid sequence encoding an enzyme with alpha-ketopimelic acid decarboxylase activity.

The invention will now be illustrated by the following examples.

### EXAMPLES

### Example 1

### General Methods

Molecular and Genetic Techniques

Standard genetic and molecular biology techniques are generally known in the art and have been previously described (Maniatis et al. 1982 “Molecular cloning: a laboratory manual”. Cold Spring Harbor Laboratory, Cold Spring Harbor, N.Y.; Miller 1972 “Experiments in molecular genetics”, Cold Spring Harbor Laboratory, Cold Spring Harbor; Sambrook and Russell 2001 “Molecular cloning: a laboratory manual” (3rd edition), Cold Spring Harbor Laboratory, Cold Spring Harbor Laboratory Press; F. Ausubel et al, eds., “Current protocols in molecular biology”, Green Publishing and Wiley Interscience, New York 1987).

Plasmids and Strains

pMS470 (Balzer, D.; Ziegelin, G.; Pansegrau, W.; Kruft, V.; Lanka, E. Nucleic Acids Research 1992, 20(8), 1851-1858.) and pBBR1MCS (Kovach M E, Phillips R W, Elzer P H, Roop R M 2nd, Peterson K M. Biotechniques. 1994 May; 16(5):800-2. pBBR1MCS: a broad-host-range cloning vector) have been described previously. E. coli strains TOP10 and DH10B (Invitrogen, Carlsbad, Calif., USA) were used for all cloning procedures. E. coli strains BL21 A1 (Invitrogen, Carlsbad, Calif., USA) and BL21 (Novagen (EMD/Merck), Nottingham, UK) were used for protein expression.

pRS414, pRS415 and pRS416 (Sikorski, R. S, and Hieter, P. A system of shuttle vectors and yeast host strains designed for efficient manipulation of DNA in Saccharomyces cerevisiae Genetics 122 (1), 19-27 (1989); Christianson, T. W., Sikorski, R. S., Dante, M., Shero, J. H. and Hieter, P. Multifunctional yeast high-copy-number shuttle vectors. Gene 110 (1), 119-122 (1992)) were used for expression in S. cerevisiae. S. cerevisiae strains CEN.PK 113-6B (ura3, trp1, leu2, MATa), CEN.PK 113-5D (ura3, MATa), CEN.PK 102-3A (ura3, leu2, MATa) and CEN.PK 113-9D (ura3, trp1, MATa) were used for protein expression.

Media

2×TY medium (16 g/l tryptopeptone, 10 g/l yeast extract, 5 g/l NaCl) was used for growth of E. colit. Antibiotics (100 μg/ml ampicillin, 50-100 μg/ml neomycin) were supplemented to maintain plasmids in E. colit. For induction of gene expression in E. colit arabinose (for BL21-Al derivatives) and IPTG (for pMS470, pBBR1MCS derivatives) were used at 0.02% (arabinose) and 0.2 mM (IPTG) final concentrations. AKP production by E. colit was done in M9 minimal medium (12.8 g/L Na₂HPO₄.7H₂O, 3 g/L KH₂PO₄, 0.5 g/L NaCl, 1 g/L NH₄Cl, 2 mM MgSO₄, 0.1 mM CaCl₂) with glucose (1-4%) or glycerol (1-4%) as carbon source, as further specified below.

Verduyn medium with 4% galactose was used for growth of S. cerevisiae.

Identification of Plasmids

Plasmids carrying the different genes were identified by genetic, biochemical, and/or phenotypic means generally known in the art, such as resistance of transformants to antibiotics, PCR diagnostic analysis of transformant or purification of plasmid DNA, restriction analysis of the purified plasmid DNA or DNA sequence analysis. Integrity of all new constructs described was confirmed by restriction digest and, if PCR steps were involved, additionally by sequencing.

UPLC-MS/MS Analysis Method for the Determination of α-Keto Acids, 6-ACA, AAP, 5-FVA and Homo₍ₙ₎Citrate

A Waters HSS T3 column 1.8 μm, 100 mm*2.1 mm was used for the separation of alpha-keto acids, 6-ACA, AAP, 5-FVA and homo(n)citrate with gradient elution as depicted in Table 1. Eluens A consists of LC/MS grade water, containing 0.1% formic acid, and eluens B consists of acetonitrile, containing 0.1% formic acid. The flow-rate was 0.25 ml/min and the column temperature was kept constant at 40° C.

TABLE 1
gradient elution program used for the separation
α-keto acids, 6-ACA, 5-FVA AAP and homo₍ₙ₎citrate

| Time (min) | 0 | 5.0 | 5.5 | 10 | 10.5 | 15 |
|---|---|---|---|---|---|---|
| % A | 100 | 85 | 20 | 20 | 100 | 100 |
| % B | 0 | 15 | 80 | 80 | 0 | 0 |

A Waters micromass Quattro micro API was used in electrospray either positive or negative ionization mode, depending on the compounds to be analyzed, using multiple reaction monitoring (MRM). The ion source temperature was kept at 130° C., whereas the desolvation temperature is 350° C., at a flow-rate of 500 L/hr.

For AKG, AKA, AKP, 5-FVA, homo-citrate and homo2-citrate the deprotonated molecule was fragmented with 10-14 eV, resulting in specific fragments from losses of e.g. H₂O, CO and CO₂.

For 6-ACA and AAP the protonated molecule was fragmented with 13 eV, resulting in specific fragments from losses of H₂O, NH₃ and CO.

To determine concentrations, a calibration curve of external standards of synthetically prepared compounds was run to calculate a response factor for the respective ions. This was used to calculate the concentrations in samples. Samples were diluted appropriately (2-10 fold) in eluent A to overcome ion suppression and matrix effects.

### Example 2

### Production of AKP by E. coli

Construction of an AKP Biosynthetic Pathway

Protein sequences for the Methanococcus jannaschii proteins homocitrate synthase (AksA, MJ0503, [Sequence ID 4]), homoaconitase small subunit (AksE, MJ1271, [Sequence ID 24]), homoaconitase large subunit (AksD, MJ1003, [Sequence ID 14]) and homoisocitrate dehydrogenase (AksF, MJ1596, [Sequence ID 34]), homologues thereof from Methanococcus manpaludis C5 (homocitrate synthase (AksA, MmarC5_{—}1522, [Sequence ID 7]), homoaconitase small subunit (AksE, MmarC5 1257, [Sequence ID 27]), homoaconitase large subunit (AksD, MmarC5 0098, [Sequence ID 17]) and homoisocitrate dehydrogenase (AksF, MmarC5 0688, [Sequence ID 37]), and A. vinelandii homocitrate synthase NifV, [Sequence ID 75]) were retrieved from databases.

M. jannaschii and M. maripaludis genes were codon pair optimized for E. coli (using methodology described in WO08000632) and constructed synthetically (Geneart, Regensburg, Germany). In the optimization procedure internal restriction sites were avoided and common restriction sites were introduced at the start and stop to allow subcloning in expression vectors. Also, upstream of AksD the sequence of the tac promoter from pMS470 was added. Each ORF was preceded by a consensus ribosomal binding site and leader sequence to drive translation in pMS470. Also, upstream of AksD the sequence of the tac promoter from pMS470 was added. A synthetic AksA [M. jannashii Sequence ID 167, M. manpaludis Sequence ID 177]/AksF [M. jannashii Sequence ID 168, M. manpaludis Sequence ID 178] cassette was cut with NdeI/XbaI and a synthetic AksD [M. jannashii Sequence ID 169, M. manpaludis Sequence ID 179]/AksE [M. jannashii Sequence ID 170, M. manpaludis Sequence ID 180] cassette was cut with XbaI/HindIII. Fragments containing Aks genes from M. jannashii were inserted in the NdeI/HindIII sites of pMS470 to obtain vector pAKP-180. Fragments containing Aks genes from M. manpaluids were inserted in the NdeI/HindIII sites of pMS470 to obtain vector pAKP-182.

An E. coli expression construct (pDB555) containing NifV from Azotobacter vinelandii [Sequence ID 149] was obtained from D. Dean (Zheng L, White R H, Dean D R. Purification of the Azotobacter vinelandii nifV-encoded homocitrate synthase. J. Bacteriol. 1997 September; 179(18):5963-6). The nifV gene was PCR amplified using phusion DNA polymerase (Finnzymes) from this vector using primers Avine-WT-R-BamHI [Sequence ID 150] and Avine-WT-F-SacI [Sequence ID 151] and cloned in pAKP-180 upstream of AksA with BamHI/SacI resulting in vector pAKP-281 [ ]. The nifV gene was also PCR amplified from this vector using primers Avine-WT-R-HindIII [Sequence ID 152] and Avine-WT-F-HindIII [Sequence ID 153] and cloned in pAKP-180 and pAKP-182 downstream of AksE [Sequence ID 170] with HindIII resulting in vector pAKP-279 and pAKP-280, respectively.

To inactivate the aksA gene in pAKP279 and pAKP281, respectively the plasmids were digested with BamHI and Bg/II resulting in three fragments (566 bps, 1134 bps, and 7776 bps). The 1134 bps and 7776 bps sized fragments were isolated from agarose gels and ligated with each other. After transformation to E. coli plasmids were checked for orientation and plasmids in which both fragments are oriented the same way as in the original plasmids pAKP279 and pAKP281 were selected resulting in pAKP322 and pAKP323, respectively.

Protein Expression and Metabolite Production in E. Coli

Plasmids pAKP-279, pAKP-280, pAKP-281, pAKP-322 and pAKP-323 were transformed to E. coli BL21 for expression. Starter cultures were grown overnight in tubes with 10 ml 2*TY medium. 200 μl culture was transferred to shake flasks with 20 ml 2*TY medium. Flasks were incubated in an orbital shaker at 30° C. and 280 rpm. After 4h IPTG was added at a final concentration of 0.2 mM and flasks were incubated for 4-16 h at 30° C. and 280 rpm. Cells from 20 ml culture were collected by centrifugation and resuspended in 4 ml M9 medium with a suitable carbon source in 24 well plates. After incubation for 24-72 h at 30-37° C. and 210 rpm cells were collected by centrifugation and pellet and supernatant were separated and stored at −20 C for analysis.

Preparation of Cell Fraction for Analysis

Cells from small scales growth (see previous paragraph) were harvested by centrifugation. The cell pellets were resuspended in 1 ml of 100% ethanol and vortexed vigorously. The cell suspension was heated for 2 min at 95° C. and cell debris was removed by centrifugation. The supernatant was evaporated in a vacuum dryer and the resulting pellet was dissolved in 200 μl deionized water. Remaining debris was removed by centrifugation and the supernatant was stored at −20° C.

Analysis of Supernatant and Cell Extract

Supernatant and extracts from cell fraction were diluted 5 times with water prior to HPLC-MS/MS analysis. Results clearly show presence of AKP and AAP in recombinant strains. It is contemplated that the conversion of AKP to AAP is catalyzed by a natural aminotransferase present in E. coli.

TABLE 2
AKP production with glucose or glycerol as carbon source

| Plasmid | Fraction | Carbon source | AKP [mg/l] | AAP [mg/l] |
|---|---|---|---|---|
| pAKP-279 | supernatant | Glucose | 3 | n.d. |
| pAKP-279 | cell | Glucose | 2 | n.d. |
| pAKP-281 | supernatant | Glucose | 3 | n.d. |
| pAKP-281 | cell | Glucose | 2 | n.d. |
| pAKP-280 | supernatant | Glucose | 2 | n.d. |
| pAKP-322 | supernatant | Glucose | 10 | 3 |
| pAKP-322 | cell | Glucose | 8 | 12 |
| pAKP-323 | supernatant | Glucose | 7 | 3 |
| pAKP-323 | cell | Glucose | 7 | 1 |
| — | supernatant | Glucose | n.d. | n.d. |
| — | cell | Glucose | n.d. | n.d. |
| pAKP-281 | supernatant | glycerol | 12 | 1 |
| pAKP-281 | cell | glycerol | 6 | 4 |
| pAKP-322 | supernatant | glycerol | 57 | 5 |
| pAKP-322 | cell | glycerol | 8 | 12 |
| pAKP-323 | supernatant | glycerol | 47 | 4 |
| pAKP-323 | cell | glycerol | 4 | 7 |
| — | supernatant | glycerol | n.d. | n.d. |
| — | cell | glycerol | n.d. | n.d. |

n.d. = not detectible

Results clearly show presence of AKP and AAP in recombinant strains. It is contemplated that the conversion of AKP to AAP is catalyzed by a natural aminotransferase present in E. coli. Removing AksA from the constructs has a positive effect on the amount of AKP and AAP produced.

### Example 3

### Production of AKP by S. cerevisiae

Construction of an Akp Biosynthetic Pathway

M. jannaschii genes were codon pair optimized for S. cerevisiae (using methodology described in WO08000632). Promoter and terminator sequences were retrieved from the S. cerevisiae genome database (www.yeastdenome.org, as available on Mar. 31, 2008). The T at position −5 in the tpi1 promoter was changed to A to generate a consensus kozak sequence for S. cerevisiae. Promoter-gene-terminator cassettes were made synthetically (Geneart, Regensburg, Germany), as shown in Table 3.

TABLE 3
Promoter-gene-terminator cassettes

| Promoter | Gene | Terminator |
|---|---|---|
| tdh1 | MJ0503 [Sequence ID 171] | tdh1 |
| tpi1 | MJ1003 [Sequence ID 172] | tpi1 |
| eno1 | MJ1271 [Sequence ID 173] | eno1 |
| tdh3 | MJ1596 [Sequence ID 174] | tdh3 |

In the optimization procedure internal restriction sites were avoided and common restriction sites were introduced at the beginning and end to allow subcloning in expression vectors.

The synthetic AksA cassette was cut with SaII/EcoRI and the synthetic AksF cassette was cut with EcoRI/XbaI and both fragments were ligated to pRS415 to obtain pAKP-136. Similarly synthetic AksD and AksE cassettes were inserted into pRS416 to obtain pAKP-146. The AksA-AksF cassette from pAKP-136 was digested with XhoI/KpnI and inserted in pAKP-146 resulting in pAKP-141. Analogous constructs were synthetically made which have a 207 bp sequence encoding a mitochondrial signal peptide (mtSP) [Sequence ID 158] N-terminally fused to MJ0503, MJ1271, MJ1003 and MJ1596 (Pfanner N, Neupert W. Distinct steps in the import of ADP/ATP carrier into mitochondria. J Biol. Chem. 1987 Jun. 5; 262(16):7528-36.). Synthetic fragments consisting of a promoter-mtSP-gene-terminator were combined in pRS416 to obtain pAKP-140. nifVwas PCR amplified from pDB555 using Phusion DNA polymerase with primers AksA-Avine-F [Sequence ID 154] and AksA-Avine-R1 [Sequence ID 155]. The gal2 promoter was amplified from pAKP-47 using phusion DNA polymerase with primers Pga12-F2 [Sequence ID 156] and Pga12-R [Sequence ID 157]. Both PCR fragments were fused by PCR using Phusion DNA polymerase and primers Pga12-F2 [Sequence ID 153] and AksA-Avine-R1 [Sequence ID 155] and the resulting fusion product was cloned in pAKP-47 with HpaI/AscI resulting in pPga12-nifV-Ttdh1. The pPga12-nifV-Ttdh1 cassette was removed from this construct by KpnI/SpeI and inserted into KpnI/SpeI digested pAKP-140 and pAKP-141 replacing MJ0503 (AksA) [Sequence ID 167] and resulting in constructs pAKP-305 and pAKP-306 respectively.

Construction of an AKP Producing S. cerevisiae Strain

S. cerevisiae strain CEN.PK113-5D was transformed with 1 μg of pAKP-305 or pAKP-306 plasmid DNA according to the method as described by Gietz and Woods (Gietz, R.D. and Woods, R.A. (2002). Transformation of yeast by the Liac/SS carrier DNA/PEG method. Methods in Enzymology 350: 87-96). Cells were plated on agar plates with 1× Yeast Nitrogen Base without amino acids and 2% glucose.

Production of AKP with S. cerevisiae

For production of AKP, starter cultures were aerobically grown overnight in 10 ml tubes containing Verduyn medium with 4% galactose at 30° C. and 280 rpm. Cultures were diluted to an OD of 0.5 in 25 ml fresh Verduyn medium with 4% galactose and incubated anaerobically and aerobically at 30° C. and 280 rpm for 2 and 5 days (aerobic cultures) an 4 days (anaerobic cultures). Cells were harvested by centrifugation and supernatant and cell fraction samples were prepared for HPLC-MS/MS analysis as described for E. coli in the Example 2.

TABLE 4
Results

| Plasmid | Fraction | AKP [mg/l] |
|---|---|---|
| pAKP305 | Supernatant | 1 |
| pAKP305 | Cell | 2 |
| pAKP306 | Supernatant | 1 |

### Example 4

### Cloning of Target Genes for Aminotransferases and Decarboxylases

Design of Expression Constructs

attB sites were added to all genes upstream of the ribosomal binding site and start codon and downstream of the stop codon to facilitate cloning using the Gateway technology (Invitrogen, Carlsbad, Calif., USA).

Gene Synthesis and Construction of Plasmids

Synthetic genes were obtained from DNA2.0 and codon optimised for expression in E. coli according to standard procedures of DNA2.0. The aminotransferase genes from Vibrio fluvialis JS17 [SEQ ID No. 1] and Bacillus weihenstephanensis KBAB4 [SEQ ID No. 82] encoding the amino acid sequences of the V. fluvialis JS17 ω-aminotransferase [SEQ ID No. 2] and the B. weihenstephanensis KBAB4 aminotransferase (ZP_{—}01186960) [SEQ ID No. 83], respectively, were codon optimised and the resulting sequences [SEQ ID No. 3] and [SEQ ID No. 85] were obtained by DNA synthesis.

The genes from Escherichia coli [SEQ ID No. 105], Saccharomyces cerevisiae [SEQ ID No. 108], Zymomonas mobilis [SEQ ID No. 111], Lactococcus lactis [SEQ ID No. 114], [SEQ ID No. 117], and Mycobacterium tuberculosis [SEQ ID No. 120] encoding the amino acid sequences of the V. fluvialis JS17 w-aminotransferase [SEQ ID No. 3], the B. weihenstephanensis KBAB4 aminotransferase (ZP_{—}01186960) [SEQ ID No. 84], the Escherichia coli diaminopimelate decarboxylase LysA [SEQ ID No. 106], the Saccharomyces cerevisiae pyruvate decarboxylase Pdc [SEQ ID No. 109], the Zymomonas mobilis pyruvate decarboxylase Pdc1472A [SEQ ID No. 112], the Lactococcus lactis branched chain alpha-keto acid decarboxylase KdcA [SEQ ID No. 115] and alpha-ketoisovalerate decarboxylase KivD [SEQ ID No. 118], and the Mycobacterium tuberculosis alpha-ketoglutarate decarboxylase Kgd [SEQ ID No. 121], respectively, were also codon optimised and the resulting sequences [SEQ ID No. 107], [SEQ ID No. 110], [SEQ ID No. 63], [SEQ ID No. 116], [SEQ ID No. 119], and [SEQ ID No. 122] were obtained by DNA synthesis, respectively.

The gene constructs were cloned into pBAD/Myc-His-DEST expression vectors using the Gateway technology (Invitrogen) via the introduced attB sites and pDONR201 (Invitrogen) as entry vector as described in the manufacturer's protocols (www.invitrogen.com). This way the expression vectors pBAD-Vfl_AT, pBAD-Bwe_AT pBAD-LysA, pBAD-Pdc, pBAD-Pdc1472A, pBAD-kdcA and pBAD-kivD were obtained, respectively The corresponding expression strains were obtained by transformation of chemically competent E. coli TOP10 (Invitrogen) with the respective pBAD-expression vectors.

Cloning by PCR

Various genes encoding a biocatalyst were amplified from genomic DNA by PCR using PCR Supermix High Fidelity (Invitrogen) according to the manufacturer's specifications, using primers as listed in the following table.

TABLE 5
overview of primers used for the various genes

| | gene | enzyme | primer |
|---|---|---|---|
| | Sequence | Sequence | Sequence |
| origin of gene | ID | ID | ID's |
| Pseudomonas aeruginosa | 85 | 86 | 87&88 |
| Pseudomonas aeruginosa | 101 | 102 | 135&136 |
| Pseudomonas aeruginosa | 141 | 142 | 147&148 |
| Pseudomonas aeruginosa | 143 | 144 | 149&150 |
| Bacillus subtilis | 89 | 90 | 123&124 |
| Bacillus subtilis | 91 | 92 | 125&126 |
| Bacillus subtilis | 139 | 140 | 145&146 |
| Rhodobacter sphaeroides | 93 | 94 | 127&128 |
| Legionella pneumophilia | 95 | 96 | 129&130 |
| Nitrosomas europaea | 97 | 98 | 131&132 |
| Neisseria gonorrhoeae | 99 | 100 | 133&134 |
| Rhodopseudomonas palustris | 103 | 104 | 137&138 |

PCR reactions were analysed by agarose gel electrophoresis and PCR products of the correct size were eluted from the gel using the QIAquick PCR purification kit (Qiagen, Hilden, Germany). Purified PCR products were cloned into pBAD/Myc-His-DEST expression vectors using the Gateway technology (Invitrogen) via the introduced attB sites and pDONR-zeo (Invitrogen) as entry vector as described in the manufacturer's protocols. The sequence of genes cloned by PCR was verified by DNA sequencing. This way the expression vectors pBAD-Pae-_gi9946143_AT, pBAD-Bsu_gi16078032 AT, pBAD-Bsu_gi16080075 AT, pBAD-Bsu_gi1607799_AT, pBAD-Rsp_AT, pBAD-Lpn_AT, pBAD-Neu_AT, pBAD-Ngo_AT, pBAD-Pae_gi9951299_AT, pBAD-Pae gi9951072_AT, pBAD-Pae_gi9951630_AT and pBAD-Rpa_AT were obtained. The corresponding expression strains were obtained by transformation of chemically competent E. coli TOP10 (Invitrogen) with the pBAD constructs.

### Example 5

### Growth of E. coli for Protein Expression

Small scale growth was carried out in 96-deep-well plates with 940 μl media containing 0.02% (w/v) L-arabinose. Inoculation was performed by transferring cells from frozen stock cultures with a 96-well stamp (Kühner, Birsfelden, Switzerland). Plates were incubated on an orbital shaker (300 rpm, 5 cm amplitude) at 25° C. for 48 h. Typically an OD_{620 nm} of 2-4 was reached.

### Example 6

### Preparation of Cell Lysates

Preparation of Lysis Buffer

The lysis buffer contained the following ingredients:

TABLE 6
lysis buffer

| | | |
|---|---|---|
| 1M MOPS pH 7.5 | 5 | ml |
| DNAse I grade II (Roche) | 10 | mg |
| Lysozyme | 200 | mg |
| MgSO₄•7H₂O | 123.2 | mg |
| dithiothreitol (DTT) | 154.2 | mg |

| | |
|---|---|
| H₂O (MilliQ) | Balance to 100 ml |

The solution was freshly prepared directly before use.

Preparation of Cell Free Extract by Lysis

Cells from small scales growth (see previous paragraph) were harvested by centrifugation and the supernatant was discarded. The cell pellets formed during centrifugation were frozen at −20° C. for at least 16 h and then thawed on ice. 500 μl of freshly prepared lysis buffer were added to each well and cells were resuspended by vigorously vortexing the plate for 2-5 min. To achieve lysis, the plate was incubated at room temperature for 30 min. To remove cell debris, the plate was centrifuged at 4° C. and 6000 g for 20 min. The supernatant was transferred to a fresh plate and kept on ice until further use.

Preparation of Cell Free Extract by Sonification

Cells from medium scales growth (see previous paragraph) were harvested by centrifugation and the supernatant was discarded. 1 ml of potassium phosphate buffer pH7 was added to 0.5 g of wet cell pellet and cells were resuspended by vigorously vortexing. To achieve lysis, the cells were sonicated for 20 min. To remove cell debris, the lysates were centrifuged at 4° C. and 6000 g for 20 min. The supernatant was transferred to a fresh tube and frozen at −20° C. until further use.

### Example 7

### Preparation of 5-Formylpentanoic Acid by Chemical Hydrolysis of Methyl 5-formylpentanoate

The substrate for the aminotransferase reaction i.e. 5-formylpentanoic acid was prepared by chemical hydrolysis of methyl 5-formylpentanoate as follows: a 10% (w/v) solution of methyl 5-formylpentanoate in water was set at pH 14.1 with NaOH. After 24 h of incubation at 20° C. the pH was set to 7.1 with HCl.

### Example 8

### Enzymatic Reactions for Conversion of 5-Formylpentanoic Acid to 6-ACA

Unless specified otherwise, a reaction mixture was prepared comprising 10 mM 5-formylpentanoic acid, 20 mM racemic α-methylbenzylamine, and 200 μM pyridoxal 5′-phosphate in 50 mM potassium phosphate buffer, pH 7.0. 100 μl of the reaction mixture were dispensed into each well of the well plates. To start the reaction, 20 μl of the cell free extracts were added, to each of the wells. Reaction mixtures were incubated on a shaker at 37° C. for 24 h. Furthermore, a chemical blank mixture (without cell free extract) and a biological blank (E. coli TOP10 with pBAD/Myc-His C) were incubated under the same conditions. Samples were analysed by HPLC-MS. The results are summarized in the following table.

TABLE 7
6-ACA formation from 5-FVA in the
presence of aminotransferases

| | 6-ACA concentration |
|---|---|
| Biocatalyst | [mg/kg] |
| E. coli TOP10/pBAD-Vfl_AT | 43* |
| E. coli TOP10/pBAD-Pae_AT | 930 |
| E. coli TOP10/pBAD-Pae_AT | 25* |
| E. coli TOP10/pBAD-Bwe_AT | 24* |
| E. coli TOP10/pBAD-Bsu_gi16077991_AT | 288 |
| E. coli TOP10/pBAD-Pae_gi9951072_AT | 1087 |
| E. coli TOP10/pBAD-Pae_gi9951630_AT | 92 |
| E. coli TOP10 with pBAD/ | 0.6 |
| Myc-His C (biological blank) | |
| None (chemical blank) | n.d. |

n.d.: not detectable
*method differed in that 10 μl cell free extract was used instead of 20 μl, the pyridoxal-5′-phosphate concentration was 50 μM instead of 200 μM and the reaction mixture volume in the wells was 190 μl instead of 100 μl.

It is shown that 6-ACA is formed from 5-FVA in the presence of an aminotransferase.

### Example 9

### Enzymatic Reactions for Conversion of AKP to 5-Formylpentanoic Acid

A reaction mixture was prepared comprising 50 mM AKP, 5 mM magnesium chloride, 100 μM pyridoxal 5′-phosphate (for LysA) or 1 mM thiamine diphosphate (for all other enzymes) in 100 mM potassium phosphate buffer, pH 6.5. 4 ml of the reaction mixture were dispensed into a reaction vessel. To start the reaction, 1 ml of the cell free extracts obtained by sonification were added, to each of the wells. In case of the commercial oxaloacetate decarboxylase (Sigma-Aldrich product number 04878), 50 U were used. Reaction mixtures were incubated with a magnetic stirrer at 37° C. for 48 h. Furthermore, a chemical blank mixture (without cell free extract) and a biological blank (E. coli TOP10 with pBAD/Myc-His C) were incubated under the same conditions. Samples from different time points during the reaction were analysed by HPLC-MS. The results are summarized in the following table.

TABLE 8
5-FVA formation from AKP in the presence of decarboxylases

| | 5-FVA concentration [mg/kg] | | |
|---|---|---|---|
| Biocatalyst | 3 h | 18 h | 48 h |
| E. coli TOP10/pBAD-LysA | 150 | 590 | 720 |
| E. coli TOP10/pBAD-Pdc | 1600 | 1700 | 1300 |
| E. coli TOP10/pBAD-Pdcl472A | 2000 | 2000 | 1600 |
| E. coli TOP10/pBAD-KdcA | 3300 | 2300 | 2200 |
| E. coli TOP10/pBAD-KivD | 820 | 1400 | 1500 |
| Oxaloacetate decarboxylase | n.d. | 6 | 10 |
| E. coli TOP10 with pBAD/ | n.d. | n.d. | n.d. |
| Myc-His C (biological blank) | | | |
| None (chemical blank) | n.d. | n.d. | n.d. |

n.d.: not detectable

It is shown that 5-FVA is formed from AKP in the presence of a decarboxylase.

### Example 10

### Enzymatic Reactions for Conversion of AKP to 6-ACA in Presence of Recombinant Decarboxylase

A reaction mixture was prepared comprising 50 mM AKP, 5 mM magnesium chloride, 100 μM pyridoxal 5′-phosphate (for LysA) or 1 mM thiamine diphosphate (for all other tested biocatalysts) in 100 mM potassium phosphate buffer, pH 6.5. 4 ml of the reaction mixture were dispensed into a reaction vessel. To start the reaction, 1 ml of the cell free extracts were added, to each of the wells. Reaction mixtures were incubated with a magnetic stirrer at 37° C. for 48 h. Furthermore, a chemical blank mixture (without cell free extract) and a biological blank (E. coli TOP10 with pBAD/Myc-His C) were incubated under the same conditions. Samples from different time points during the reaction were analysed by HPLC-MS. The results are summarized in the following table.

TABLE 9
6-ACA formation from AKP in the presence of decarboxylases

| | 6-ACA concentration | | |
|---|---|---|---|
| | [mg/kg] | | |
| Biocatalyst | 3 h | 18 h | 48 h |
| E. coli TOP10/pBAD-LysA | n.a. | 0.01 | 0 |
| E. coli TOP10/pBAD-Pdc | 0.1 | 0.3 | n.a. |
| E. coli TOP10/pBAD-Pdcl472A | 0.03 | 0.1 | 0.2 |
| E. coli TOP10/pBAD-KdcA | 0.04 | 0.1 | 0.3 |
| E. coli TOP10/pBAD-KivD | n.a. | 0.3 | 0.6 |
| E. coli TOP10 with pBAD/ | n.d. | n.d. | n.d. |
| Myc-His C (biological blank) | | | |
| None (chemical blank) | n.d. | n.d. | n.d. |

n.a. = not analysed
n.d. = not detectable

It is shown that 6-ACA is formed from AKP in the presence of a decarboxylase. It is contemplated that the E. coli contained natural 5-FVA aminotransferase activity.

### Example 11

### Enzymatic Reactions for Conversion of AKP to 6-ACA in Presence of Recombinant Decarboxylase and Recombinant Aminotransferase

A reaction mixture was prepared comprising 50 mM AKP, 5 mM magnesium chloride, 100 μM pyridoxal 5′-phosphate, 1 mM thiamine diphosphate and 50 mM racemic α-methylbenzylamine in 100 mM potassium phosphate buffer, pH 6.5. 1.6 ml of the reaction mixture were dispensed into a reaction vessel. To start the reaction, 0.2 ml of the decarboxylase containing cell free extract and 0.2 ml of the aminotransferase containing cell free extract were added, to each of the reaction vessels. Reaction mixtures were incubated with a magnetic stirrer at 37° C. for 48 h. Furthermore, a chemical blank mixture (without cell free extract) and a biological blank (E. coli TOP10 with pBAD/Myc-His C) were incubated under the same conditions. Samples from different time points during the reaction were analysed by HPLC-MS. The results are summarized in the following table.

TABLE 10
6-ACA formation from AKP in the presence of a recombinant
decarboxylase and a recombinant aminotransferase

| | 6-ACA concentration [mg/kg] after 48 hours | | |
|---|---|---|---|
| | AT | | |
| | E. coli TOP10/ | E. coli TOP10/ | E. coli TOP10/pBAD- |
| DC | pBAD-Vfl-AT | pBAD-Bwe-AT | PAE_gi9946143_AT |
| E. coli TOP10/ | 183.4 | 248.9 | 117.9 |
| pBAD-Pdc | | | |
| E. coli TOP10/ | 458.5 | 471.6 | 170.3 |
| pBAD-Pdcl472A | | | |
| E. coli TOP10/ | 497.8 | 497.8 | 275.1 |
| pBAD-KdcA | | | |
| E. coli TOP10/ | 510.9 | 510.9 | 314.4 |
| pBAD-KivD | | | |

AT = aminotransferase
DC = decarboxylase

In the chemical blank and in the biological blank no 6-ACA was detectable.

Further, the results show that compared to the example wherein a host-cell with only recombinant decarboxylase (and no recombinant aminotransferase) the conversion to 6-ACA was improved.

### Example 12

### Production of 6-ACA in E. coli

Preparation of Constructs for Co-Expression of Aminotransferases and a Decarboxylases

Construction of the plasmids containing genes which encode enzymes for conversion of AKP to 5-formyl valeric acid (5-FVA) and 5-FVA to 6-ACA was done as described in Example 4. To allow co-expression of an aminotransferase and a decarboxylase a tac promoter cassette was PCR amplified from pF113 (a derivative of pJF119EH (Fürste, J. P., W. Pansegrau, R. Frank, H. Blocker, P. Scholz, M. Bagdasarian, and E. Lanka. 1986. Molecular cloning of the plasmid RP4 primase region in a multi-host-range tacP expression vector. Gene 48:119-131.) which contains two NotI sites at positions 515 and 5176 respectively with the tac promoter being the start of the numbering), using Phusion DNA polymerase and primers pF113-F-NsiI (aaattatgcatACAGCATGGCCTGCAACG) and pF113-R-AgeI (aaattaccggtCAGGGTTATTGTCTCATGAG) and the resulting PCR fragment was fused to NsiI/AgeI digested pBBR1MCS (Kovach M E, Phillips R W, Elzer P H, Roop R M 2nd, Peterson K M. Biotechniques. 1994 May; 16(5):800-2. pBBR1MCS: a broad-host-range cloning vector) resulting in pBBR-lac. The aminotransferase gene from Vibrio fluvialis JS17 ((Seq ID NO:1) was codon optimised (Seq ID NO: 3). This codon optimised gene and the gene from Pseudomonas aeruginosa PA01 coding for AT-Vfl and AT-PA01 (Seq ID 85) respectively were PCR amplified from pBAD/Myc-His-DEST-AT-Vfl and pBAD/Myc-his-DEST-PA01 using Phusion DNA polymerase according to the manufacturers specifications using primer pairs AT-Vfl_for_Ec (AAATTT GGTACC GCTAGGAGGAATTAACCATG)+AT-Vfl_rev_Ec (AAATTT ACTAGT AAGCTGGGTTTACGCGACTTC) and AT-Pa01 for Ec (AAATTT GGTACC GCTAGGAGGAATTAACCATG)+AT-Pa01 rev Ec, (AAATTT ACTAGTACAAGAAAGCTGGGTTCAAG) respectively.

The decarboxylase gene from Lactococcus lactis coding for Lactococcus lactis branched chain alpha-keto acid decarboxylase KdcA (Seq ID NO: 116) was amplified from pBAD/Myc-His-DEST-DC-KdcA by PCR using Phusion DNA polymerase according to the manufacturers specifications and using primers Kdc_for_Ec (AAATTT ACTAGT GGCTAGGAGGAATTACATATG) and Kdc_rev_Ec (AAATTT AAGCTT ATTACTTGTTCTGCTCCGCAAAC). The aminotransferase fragments were digested with KpnI/SpeI and the decarboxylase fragment was digested with SpeI/HindIII. Both fragments were ligated to KpnI/HindIII digested pBBR-lac to obtain pAKP-94 (containing genes encoding AT-PA01 and KdcA) and pAKP-96 (containing genes encoding AT-Vfl and KdcA) respectively.

Protein Expression and Metabolite Production in E. Coli

Plasmid pAKP-323 (described in Example 2) was co-transformed with pAKP96 to E. coli BL21 for expression. Cultures were grown as described in Example 2. Samples were prepared for analysis as described in Example 2 and analysed by LC-MS-MS as described in Example 1.

TABLE 11

| Plas- | Plas- | C- | Incu- | me- | Culture | 6-ACA |
|---|---|---|---|---|---|---|
| mid 1 | mid 2 | source | bation | dium | condition | [μg/l] |
| pAKP-323 | pAKP-96 | glucose | 24 h | 2x | 24 wells | 18 |
| | | | | TY | MTP | |
| pAKP-323 | pAKP-96 | glycerol | 24 h | 2x | 24 wells | 109 |
| | | | | TY | MTP | |
| pAKP-323 | pAKP-96 | glucose | 24 h | M9 | 24 wells | 8 |
| | | | | | MTP | |
| pAKP-323 | pAKP-96 | glycerol | 24 h | M9 | 24 wells | 107 |
| | | | | | MTP | |
| pAKP-323 | pAKP-96 | glucose | 24 h | M9 | Shake flask | 1120 |

E. coli BL21 was either transformed with plasmid pAKP-322 (strains eAKP233), plasmid pAKP96 (Strain eAKP 71) or with plasmid pAKP94 (Strain eAKP70). Cultures were grown overnight in tubes with 10 ml 2*TY medium. 200 μl culture was transferred to shake flasks with 20 ml 2*TY medium. Flasks were incubated in an orbital shaker at 30° C. and 280 rpm. After 4 h IPTG was added at a final concentration of 0.1 mM and flasks were incubated for 4 h at 30° C. and 280 rpm. Cells from 10 ml culture were collected by centrifugation and resuspended in 10 ml M9 medium with 0.4% glucose. The two cultures were mixed in various ratios in 24 well plates and after incubation for 48-72 h at 37° C. and 210 rpm the supernatant was collected by centrifugation and stored at −20 C for analysis. Samples were prepared for analysis by LC-MS-MS as described in Example 2 and analysed as described in Example 1.

TABLE 12

| Strain | Strain | Strain | | 6-ACA |
|---|---|---|---|---|
| eAKP233 | eAKP71 | eAKP70 | Incubation | [μg/l] |
| 5 ml | 0 ml | 0 ml | 48 h | 0 |
| 4 ml | 1 ml | 0 ml | 48 h | 0 |
| 2.5 ml | 2.5 ml | 0 ml | 48 h | 26 |
| 1 ml | 4 ml | 0 ml | 48 h | 40 |
| 0 ml | 5 ml | 0 ml | 48 h | 0 |
| 5 ml | 0 ml | 0 ml | 72 h | 0 |
| 4 ml | 0 ml | 1 ml | 72 h | 68 |
| 2.5 ml | 0 ml | 2.5 ml | 72 h | 71 |
| 1 ml | 0 ml | 4 ml | 72 h | 85 |
| 0 ml | 0 ml | 5 ml | 72 h | 0 |

### Example 13

### Construction of an AKP Biosynthetic Pathway from Other Archae Bacteria

Protein sequences for the Methanosarcina activorans homoaconitase small subunit (AksE, MA3751, [Sequence ID 225]), homoaconitase large subunit (AksD, MA3085, [Sequence ID 237]) and homoisocitrate dehydrogenase (AksF, MA3748, [Sequence ID 249]), homologues thereof from Methanospirillum hungatei JF-1 homoaconitase small subunit (AksE, Mhun_{—}1799, [Sequence ID 228]), homoaconitase large subunit (AksD, Mhun_{—}1800, [Sequence ID 240]) and homoisocitrate dehydrogenase (AksF, Mhun_{—}1797, [Sequence ID 252]), homologues thereof from Methanococcus maripaludis S2 homoaconitase small subunit (AksE, MMP0381, [Sequence ID 207]), homoaconitase large subunit (AksD, MMP1480, [Sequence ID 195]) and homoisocitrate dehydrogenase (AksF, [Sequence ID 222]), homologues thereof from Methanococcus vannielii SB homoaconitase small subunit (AksE, Mevan_{—}1368, [Sequence ID 201]), homoaconitase large subunit (AksD, Mevan_{—}0789, [Sequence ID 189]) and homoisocitrate dehydrogenase (AksF, Mevan_{—}0040 [Sequence ID 216]), and A. vinelandii homocitrate synthase NifV, [Sequence ID 75]) were retrieved from databases.

TABLE 13

| | | NifV | AksD | AksE | AksF |
|---|---|---|---|---|---|
| Plasmid ID | Donor organism(s) | Seq ID | Seq ID | Seq ID | Seq ID |
| pAKP-358 | Methanosarcina acetivorans & | 149 | 236 | 224 | 248 |
| | Azotobacter vinelandii (NifV) | | | | |
| pAKP-359 | Methanospirillum hungatei JF-1 & | 149 | 239 | 227 | 251 |
| | Azotobacter vinelandii (NifV) | | | | |
| pAKP376 | Methanococcus vannielii SB & | 149 | 188 | 200 | 215 |
| | Azotobacter vinelandii (NifV) | | | | |
| pAKP378 | Methanococcus maripaludis S2& | 149 | 194 | 206 | 221 |
| | Azotobacter vinelandii (NifV) | | | | |

Genes encoding the homoaconitase small subunit (AksE), homoaconitase large subunit (AksD) and homoisocitrate dehydrogenase (AksF) were codon pair optimized for E. coli (using methodology described in WO08000632) (table 13). Constructs were made synthetically (Geneart, Regensburg, Germany) containing the optimized genes together with the wild-type nifV gene (Seq ID149). In the optimization procedure internal restriction sites were avoided and common restriction sites were introduced at the start and stop to allow subcloning in expression vectors. Also, upstream of AksD the sequence of the tac promoter from pMS470 was added. Each ORF was preceded by a consensus ribosomal binding site and leader sequence to drive translation in pMS470. Also, upstream of AksD the sequence of the tac promoter from pMS470 was added. A synthetic AksA/AksF cassette was cut with NdeI/XbaI and a synthetic AksD/AksE cassette was cut with XbaI/HindIII. Fragments containing Aks genes were inserted in the NdeI/HindIII sites of pMS470 to obtain the vectors pAKP-358, pAKP359, pAKP376 and pAKP378.

Protein Expression and Metabolite Production in E. Coli

Plasmids were transformed to E. coli BL21 for expression. Starter cultures were grown overnight in tubes with 10 ml 2*TY medium. 200 μl culture was transferred to shake flasks with 20 ml 2*TY medium. Flasks were incubated in an orbital shaker at 30° C. and 280 rpm. After 4 h IPTG was added at a final concentration of 0.2 mM and flasks were incubated for 4-16 h at 30° C. and 280 rpm. Cells from 20 ml culture were collected by centrifugation and resuspended in 4 ml M9 medium with a suitable carbon source in 24 well plates. After incubation for 24-72 h at 30-37° C. and 210 rpm cells were collected by centrifugation and pellet and supernatant were separated and stored at −20 C for analysis.

Preparation of Cell Fraction for Analysis

Cells from small scales growth (see previous paragraph) were harvested by centrifugation. The cell pellets were resuspended in 1 ml of 100% ethanol and vortexed vigorously. The cell suspension was heated for 2 min at 95° C. and cell debris was removed by centrifugation. The supernatant was evaporated in a vacuum dryer and the resulting pellet was dissolved in 200 μl deionized water. Remaining debris was removed by centrifugation and the supernatant was stored at −20° C.

Analysis of Supernatant and Cell Extract

Supernatant and extracts from cell fraction were diluted 5 times with water prior to HPLC-MS/MS analysis. Results, shown in Table 14, clearly show presence of AKP and AAP in recombinant strains. It is contemplated that the conversion of AKP to AAP is catalyzed by a natural aminotransferase present in E. coli.

TABLE 14
AKP production with glycerol as carbon source

| Plasmid | Fraction | Carbon source | AKP [mg/l] | AAP [mg/l] |
|---|---|---|---|---|
| — | supernatant | glycerol | n.d. | n.d. |
| — | cell | glycerol | n.d. | n.d. |
| pAKP358 | supernatant | glycerol | 21 | 24 |
| pAKP359 | supernatant | glycerol | 19 | 27 |
| pAKP376 | supernatant | glycerol | 3 | 0.4 |
| pAKP378 | supernatant | glycerol | 650 | 134 |

n.d. = not detectible

Results clearly show presence of AKP and AAP in recombinant strains. It is contemplated that the conversion of AKP to AAP is catalyzed by a natural aminotransferase present in E. coli.

### Example 14

### Production of 6-ACA from AKP in E. coli

Preparation of Constructs for Co-Expression of Aminotransferases and Decarboxylases

Construction of the plasmids encoding enzymes for conversion of AKP to 5-formyl valeric acid (5-FVA) and 5-FVA to 6-ACA was as described in Example 4 whereas the plasmids pAKP94 and pAKP96 were described in example 12. For exchanging the Lactococcus lactis branched chain alpha-keto acid decarboxylase KdcA [SEQ ID No. 115], present in pAKP 94 and pAKP96 with the Zymomonas mobilis pyruvate decarboxylase Pdcl472A [SEQ ID No. 112], and alpha-ketoisovalerate decarboxylase KivD [SEQ ID No. 118], respectively plasmids pBAD-kivD and pBAD-Pdc1472A were digested with Nde1 and HinD3. The 1,6 kb fragment containing the decarboxylase gene was isolated and ligated into the Nde1/HinD3 digested vector pAKP94 yielding pAKP 326 and pAKP327 respectively. Cloning the 1.6 kb Nde1/HinD3 fragments from pBAD-kivD into pAKP96 yielded pAKP330.

Protein Expression and Metabolite Production in E. Coli

Plasmids were transformed to E. coli BL21 for expression. Starter cultures were grown overnight in tubes with 10 ml 2*TY medium. 200 μl culture was transferred to shake flasks with 20 ml 2*TY medium. Flasks were incubated in an orbital shaker at 30° C. and 280 rpm. After 4 h IPTG was added at a final concentration of 0.2 mM and flasks were incubated for 4 h at 30° C. and 280 rpm. Cells from 20 ml culture were collected by centrifugation and resuspended in 4 ml 2×TY medium with 1% glycerol and 500 mg/l AKP in 24 well plates. After incubation for 48 h at 30° C. and 210 rpm cells were collected by centrifugation and pellet and supernatant were separated and stored at −20 C for analysis.

TABLE 15
6-ACA production in E. coli

| plasmid | aminotransferase | Decarboxylase | Mg/l AAP | Mg/l 6-ACA |
|---|---|---|---|---|
| pAKP326 | PA01 | kivD | 34 | 21 |
| pAKP327 | PA01 | pdcl472A | 25 | 1 |
| pAKP330 | Vfl | kivD | 18 | 18 |

Results clearly show presence of 6-ACA and AAP in recombinant strains. It is contemplated that the conversion of AKP to AAP is catalyzed by a natural aminotransferase present in E. coli.

1. Method for preparing alpha-ketopimelic acid, comprising converting alpha-ketoglutaric acid into alpha-ketoadipic acid and converting alpha-ketoadipic acid into alpha-ketopimelic acid, wherein at least one of these conversions is carried out using a heterologous biocatalyst.

2. Method according to claim 1, wherein alpha-ketoglutaric acid is biocatalytically prepared from a carbon source, in particular from a carbohydrate.

3. Method according to claim 1, wherein the heterologous biocatalyst comprises a heterologous biocatalyst catalysing C₁-elongation of alpha-ketoglutaric acid into alpha-ketoadipic acid and/or C₁-elongation of alpha-ketoadipic acid into alpha-ketopimelic acid.

4. Method according to claim 3, wherein the heterologous biocatalyst comprises
a. an AksA enzyme having homo₍ₙ₎citrate activity or an homologue thereof;
b. at least one enzyme selected from the group of AksD enzymes having homoₙ-aconitase activity, AksE enzymes having homoₙ-aconitase activity, homologues of said AksD enzymes and homologues of said AksE enzymes; and
c. an AksF enzyme having homoₙ-isocitrate dehydrogenase or a homologue thereof.

5. Method according to claim 3, wherein the heterologous enzyme system originates from an organism selected from the group of methanogenic archae, preferably selected from the group of Methanococcus, Methanocaldococcus, Methanosarcina, Methanothermobacter, Methanosphaera, Methanopyrus and Methanobrevibacter.

6. Method according to claim 1, wherein the heterologous biocatalyst comprises an enzyme system catalysing the conversion of alpha-ketoglutaric acid into alpha-ketoadipic acid, wherein said enzyme system forms part of the amino adipate pathway for lysine biosynthesis.

7. Method according to claim 6, wherein the enzyme system is from an organism selected from the group of yeasts, fungi, archaea and bacteria, in particular from the group of Penicillium, Cephalosporium, Paelicomyces, Trichophytum, Aspergillus, Phanerochaete, Emericella, Ustilago, Schizosaccharomyces, Saccharomyces, Candida, Yarrowia, Pichia, Kluyveromyces, Thermus, Deinococcus, Pyrococcus, Sulfolobus, Thermococcus, Methanococcus, Methanocaldococcus, Methanosphaera, Methanopyrus, Methanobrevibacter, Methanosarcina and Methanothermobacter.

8. Method according to claim 1, wherein the heterologous biocatalyst comprises an enzyme system catalysing the conversion of alpha-ketoglutaric acid into alpha-ketoadipic acid, wherein at least one of the enzymes of the enzyme system originates from nitrogen fixing bacteria selected from the group of cyanobacteria, rhizobiales, γ-proteobacteria and actinobacteria, in particular from the group of Anabaena, Microcystis, Synechocystis, Rhizobium, Bradyrhizobium, Pseudomonas, Azotobacter, Klebsiella and Frankia.

9. Method for preparing 5-formylpentanoic acid, comprising biocatalytically decarboxylating alpha-ketopimelic acid prepared in a method according to claim 1, thereby forming 5-formylpentanoic acid.

10. Method for preparing 6-aminocaproic acid, comprising converting 5-formylpentanoic acid, prepared in a method according to claim 9, into 6-aminocaproic acid.

11. Method according to claim 10 wherein the conversion of 5-formylpentanoic acid into 6-aminocaproic acid comprises transamination or reductive amination.

12. Method for preparing 6-aminocaproic acid, comprising converting alpha-ketopimelic acid prepared in a method according to claim 1 into alpha-aminopimelic acid and converting alpha-aminopimelic acid into 6-aminocaproic acid, which conversions are preferably carried out biocatalytically.

13. Method for preparing alpha-ketosuberic acid from alpha-ketopimelic acid prepared in a method according to claim 1 comprising subjecting the alpha-ketopimelic acid to C₁-elongation, using a biocatalyst having catalytic activity with respect to said C₁-elongation, in particular a biocatalyst comprising
d. an AksA enzyme having homo₍ₙ₎citrate activity or an homologue thereof;
e. at least one enzyme selected from the group of AksD enzymes having homoₙ-aconitase activity, AksE enzymes having homoₙ-aconitase activity, homologues of said AksD enzymes and homologues of said AksE enzymes; and
f. an AksF enzyme having homoₙ-isocitrate dehydrogenase or a homologue thereof.

14. Method according to claim 13, wherein the enzymes each indecently originate from an organism selected from the group of methanogenic archae, preferably selected from the group of Methanococcus, Methanocaldococcus, Methanosarcina, Methanothermobacter, Methanosphaera, Methanopyrus and Methanobrevibacter.

15. Method for preparing 7-aminoheptanoic acid comprising converting alpha-ketosuberic acid prepared in a method according to claim 13.

16. Method according to claim 1, wherein the method is carried out under fermentative conditions.

17. Heterologous cell, comprising one or more heterologous nucleic acid sequences encoding one or more heterologous enzymes having catalytic activity in at least one reaction step in the preparation of alpha ketopimelic acid from alpha-ketoglutaric acid.

18. Heterologous cell according to claim 17, wherein the cell is free of aminotransferases capable of catalysing the conversion of alpha-ketoadipate into alpha-aminoadipate.

19. Heterologous cell according to claim 17, comprising at least one nucleic acid sequence encoding an enzyme represented by any of the sequence ID's 4-77 261, 264, 267, 270, 273, 276, 279, 282, 186, 189, 192, 195, 225, 228, 231, 234, 198, 201, 204, 207, 237, 240, 243, 246, 210, 213, 216, 219, 222, 249, 252, 255, 258 or a homologue thereof.

20. Heterologous cell according to claim 17, comprising a nucleic acid sequence encoding an enzyme having catalytic activity with respect to the decarboxylation of alpha-ketopimelic acid to form 5-formylpentanoic acid, in particular such an enzyme selected from the group of decarboxylases (E.C. 4.1.1), more in particular from the group of glutamate decarboxylases (EC 4.1.1.15), diaminopimelate decarboxylases (EC 4.1.1.20) aspartate 1-decarboxylases (EC 4.1.1.11), branched chain alpha-keto acid decarboxylases, alpha-ketoisovalerate decarboxylases, alpha-ketoglutarate decarboxylases, pyruvate decarboxylases (EC 4.1.1.1), and oxaloacetate decarboxylases (E.C. 4.1.1.3).

21. Heterologous cell according to claim 17, wherein the cell is from an organism selected from the group of Penicillium chrysogenum, Aspergillus niger, Ustilago maydis, Saccharomyces cerevisiae, Kluyveromyces lactis, Pichia pastoris, Hansenula polymorha, Escherichia coil, Azotobacter vinelandii, Pseudomonas stutzerii, Klebsiella pneumoniae, Deinococcus radiourans, Deinococcus geothermalis, Thermus thermophllus, Methanococcus maripaludis, Methanosarcina acetivorans, Methanospirillum hungatei, Methanosaeta thermophile Methanobrevibacter smithii, Methanococcus vannielii, Methanococcus aeolicus and Methanocaldococcus jannashii.

22. Heterologous cell according to claim 17, comprising at least one nucleic acid sequence represented by any of the sequences selected from the group of SEQ ID NO 149; SEQ ID NO's 145, 146, 147, 148; SEQ ID NO's 167, 168, 169, 170, 171, 172, 173, 174; SEQ ID NO's 177, 178, 179, 180, 181, 182, 183, 184; SEQ ID NO's 224, 226, 236, 238, 248, 250, 260, 262; SEQ ID NO's 227, 229, 239, 241, 251, 253, 263, 265; SEQ ID NO's; 194, 196, 206, 208, 221, 223, 281, 283; SEQ ID NO's; 188, 190, 200, 202, 215, 217, 272, 274 and functional analogues thereof.

23. Use of a heterologous cell according to claim 17 in the preparation of caprolactam, 6-aminocaproic acid or diaminohexane.
